(19) United States
(12) Patent Application Publication
(10) Pub. No.: US 2026/0125669 A1
(43) Pub. Date: May 7, 2026

(54) METHODS AND COMPOSITIONS FOR SYNTHETIC EVOLUTION

(71) Applicant: UNIVERSITAT POMPEU FABRA, Barcelona (ES)

(72) Inventors: Dimitrije IVANCIC, Barcelona (ES); Marc GÜELL CARGOL, Barcelona (ES)

(73) Assignee: UNIVERSITAT POMPEU FABRA, Barcelona (ES)

(21) Appl. No.: 19/118,859

(22) PCT Filed: Oct. 6, 2023
(86) PCT No.: PCT/EP2023/077778

§ 371 Date: Apr. 7, 2025

(30) Foreign Application Priority Data
Oct. 7, 2022 (EP) 22200207.3

(51) Int. Cl. C12N 15/10; C12N 15/86

(52) U.S. Cl. CPC C12N 15/1058; C12N 15/86; C12N 2740/15043; C12N 2740/15052

(57) ABSTRACT

A method of synthetic evolution of a nucleic acid sequence of interest and the mutant nucleic acid sequence of interest obtained therefrom. The method utilizes a virus-assisted evolution platform for hyper-directed evolution of phenotypes in mammalian cells. The virus-assisted evolution platform includes co-transfected mammalian producer cells with at least a first nucleic acid sequence encoding a Retroviridae genome including at least the gag and pol genes and a at least a second nucleic acid encoding a recombinant expression cassette.

### FIELD OF INVENTION

The present invention relates to the field of mammalian gene synthetic evolution.

### BACKGROUND OF INVENTION

Evolution is the main architect of the existing biological world. However, protein optimization for biotherapeutic use requires tough testing of multiple variants.

Scientists have coined the term “synthetic evolution” to refer to the application of modern molecular and synthetic biology approaches to iteratively diversify and select one or more targeted genetic loci with desired functions or phenotypes. This ability to accelerate evolution ultimately allows the ‘hyperdirected’ evolution of phenotypes never before seen in nature.

Recreating evolution in the laboratory has been successfully used in the past to generate new protein functions with therapeutic use, such as antibodies (Jespers et al., 1994. Biotechnology (N Y). 12(9):899-903) or anti-tumor agents (Swe et al., 2012. Biochem Pharmacol. 84(6):775-783).

More recently, tools and platforms for synthetic evolution have been developed (Simon et al., 2019. Nat Biotechnol. 37(7):730-743; US20030175972 A1; Rossolillo et al., 2012. PLOS Genetics; Yenerall et al., 2021. Cancer Research). In particular, virus-assisted evolution has been developed to trigger the synthetic evolution of individual genes or subset of genes. For instance, the team of David Liu at Harvard has developed “PACE”, for “phage-assisted continuous evolution”, using M13 bacteriophages containing a gene of interest to be evolved, to infect Escherichia coli cells and propagate from one cell to another. With each cycle of infection and propagation, the phage's nucleic acid including the gene of interest is subject to mutations, and phage growth enables the selection of the most active variants (Esvelt et al., 2011. Nature. 472(7344):499-503; WO 2010/028347 A2; WO 2012/088381 A2).

The PACE platform, with its use of a M13 phage, is however limited to prokaryotic systems and has been mainly used for bacterial protein evolution (Badran et al., 2016. Nature. 533(7601):58-63; Bryson et al., 2018. Nat Chem Biol. 13(12):1253-1260; Hu et al., 2018. Nature. 556(7699):57-63).

PACE-like methods have then been adapted to evolve mammalian genes, for instance, by coupling the replication of an adenovirus engineered to contain a mammalian gene of interest but to lack an essential protease, which is supplied in trans in mammalian cells dependently on the activity of the target gene (Berman et al., 2018. J Am Chem Soc. 140(51):18093-18103; WO 2019/084384 A1). More recently, a Sindbis RNA Alphavirus was used as vector in the “VEGAS” platform (Viral Evolution of Genetically Actuating Sequences) to evolve GPCRs and transcription factors (English et al., 2019. Cell. 178(3):748-761.e17).

However, these methods and platforms are not devoid of drawbacks: first, they rely on hyper-mutagenic viruses (for instance, the Sindbis RNA Alphavirus is estimated to have a mutagenesis rate of about 10-3 to 10-4 substitutions/nucleotide/cell infection) which makes it difficult if not impossible to control mutagenesis as well as mutation spectrum (e.g., transversion/transition substitution ratio) that can occur. Indeed, given the very high mutagenesis rate of these viruses, each iteration of the method introduces not one or a few, but several mutations in the gene of interest (e.g., up to 10 mutations in a 10-kb sequence with a substitution rate of 10-3), possibly leading in a majority of cases to the co-creation of deleterious mutations and ultimately, the generation of non-functional products, which obscures the detection of beneficial mutations. It requires thus a significant amount of trials-and-error, to obtain an evolved sequence of interest with functional and advantageous mutations.

Second, the viruses used in the existing platforms do not exhibit recombination events, which would however add combinatorial diversity in the process; they also do not integrate in a host cell's genome, although this may be desirable for high expression of the sequence of interest and ultimately, an easier selection of the evolved sequences of interest with a desired biological activity.

Third, these methods and platforms do not allow to target a specific region of the sequence of interest to be mutated. It would however be desirable to have a method which allows to select, within a full sequence of interest, a specific region to be evolved (for instance, a region coding for a given functional domain of a gene's product).

Here, we have developed a “Retroviral Synthetic Evolution” platform or “RSE” platform, the first directed evolution platform that can screen billions of protein variants in mammalian systems cost-efficiently. The platform works on the principle of iterative cycles of (i) in vivo diversification, where millions of variants of a sequence of interest are produced; (ii) in vivo encapsulation, where these variants are encoded in biological nanoparticles; and (iii) in vivo screening, where the biological nanoparticles are delivered to reporter mammalian cells. Upon delivery to the mammalian cells, the sequence of interest's variant produces a measurable phenotype and the top variants, i.e., those exhibiting the desired phenotype, can be selected for further iterations of the cycle.

The platform makes use of retroviruses, which mutagenesis rate makes it possible to tune the evolution process more finely, by introducing only very few mutations, possibly only one, at each iteration of the method. In addition, mutagens are used with this method to control the mutation rate on demand. Retroviruses also exhibit recombination events, and can integrate in a host cell's genome, adding diversity to the evolution process and allowing for an easy selection of the evolved sequences of interest in reporter cells. Finally, using programmable RNA-guided proteins, the RSE platform allows to target specific regions to be evolved within a gene of interest.

### SUMMARY

The present invention relates to a method of synthetic evolution of a nucleic acid sequence of interest (SoI), comprising:
(a) co-transfecting mammalian producer cells with
   (i) a first nucleic acid sequence encoding a Retroviridae genome comprising at least the gag and pol genes,
   (ii) a second nucleic acid sequence encoding a recombinant expression cassette comprising, from 5′ to 3′:
      a 5′ long terminal repeat (5′ LTR),
      a packaging sequence,
      the SoI to be evolved,
      a 3′ long terminal repeat (3′ LTR); and
   (iii) optionally, a third nucleic acid sequence encoding a viral envelope glycoprotein;
(b) incubating the mammalian producer cells under conditions allowing for mutation of the SoI and production of Retroviridae vectors;
(c) optionally, harvesting the population of Retroviridae vectors produced after step (b);
(d) infecting mammalian reporter cells with the population of Retroviridae vectors of (c);
(e) incubating the mammalian reporter cells under conditions allowing for SoI expression; and
(f) selecting one or several mutants of the SoI with a desired biological activity;
wherein steps (a)-(f) are reiterated until a mutant of the SoI with the desired biological activity is obtained.

In some embodiments, mammalian producer cells are incubated at step (b) in the presence of a mutagen.

In some embodiments, the mutagen is a nucleoside analog. In some embodiments, the nucleoside analog is selected from the group consisting of 5-hydroxy-2′-deoxycytidine (5-OH-dC) and 5-azacytidine (5-aza-C).

In some embodiments, mammalian producer cells are incubated at step (b) in the presence of a single-stranded or double-stranded RNA-specific deaminase or a nucleic acid coding therefor. In some embodiments, the RNA-specific deaminase is an RNA-specific adenosine deaminase or an RNA-specific cytidine deaminase.

In some embodiments, the deaminase is fused to a programmable RNA-guided protein and the mammalian producer cells are further incubated at step (b) with a guide RNA comprising a first region at least partially complementary to a region of interest of the SoI and a second region capable of interacting with the programmable RNA-guided protein.

In some embodiments, the pol gene expresses an error-prone reverse transcriptase/RNaseH mutant. In some embodiments, the error-prone reverse transcriptase/RNaseH mutant comprises at least one mutation selected from the group consisting of Y115A, M184V, M184I, Q151M, M230I and Y501W, said position number corresponding to the amino acid number of SEQ ID NO: 1.

In some embodiments, the Retroviridae vector is a lentiviral vector, preferably selected from the group consisting of human immunodeficiency virus 1 (HIV-1), human immunodeficiency virus 1 (HIV-2), simian immunodeficiency virus (SIV), feline immunodeficiency virus (FIV), bovine immunodeficiency virus (BIV), puma Lentivirus (PLV), equine infectious anemia virus (EIAV), caprine arthritis encephalitis virus (CAEV), Visna-maedi virus, and Jembrana disease virus, preferably the Retroviridae vector is HIV-1 or HIV-2.

In some embodiments, the Retroviridae vectors are not self-inactivating vectors.

In some embodiments, the mammalian producer cells and/or the mammalian reporter cells are human cells, preferably HEK 293T cells.

In some embodiments, the SoI is a coding nucleic acid sequence or a non-coding nucleic acid sequence. In some embodiments, the SoI is a regulatory element or expresses a non-coding RNA. In some embodiments, the SoI is a coding nucleic acid sequence encoding a peptide or protein.

In some embodiments, selection of mutants of the SoI at step (f) is performed based on a measurable phenotype directly or indirectly caused by the SoI mutant in the mammalian reporter cells.

In some embodiments, the mammalian reporter cells at steps (d) and (e) express an exogenous protein or fragment thereof acting as reporter for the selection of mutants of the SoI with a desired biological activity.

### Definitions

In the present invention, the following terms have the following meanings.

“Fusion protein” refers to a protein having at least two heterologous polypeptides covalently linked either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion proteins may be fused in any order. This term also refers to conservatively modified variants, polymorphic variants, alleles, mutants, subsequences, and interspecies homologs of the antigens that make up the fusion protein.

“Identity” or “identical”, when used in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”). Identity of related amino acid sequences or nucleic acid sequences can be readily calculated by known methods. Preferred methods for determining identity are designed to give the largest match between the sequences tested. Methods of determining identity are described in publicly available computer programs.

“Integration” refers to the addition of a nucleic acid sequence into a second nucleic acid sequence, or into a genome or a portion thereof.

“Linker” refers to a chemical group or a molecule linking two adjacent molecules or moieties.

“Modified” refers to a protein or nucleic acid sequence that is different than a corresponding unmodified protein or nucleic acid sequence.

“Mutagen” refers to a compound or process that results in the introduction of one or more mutations in a nucleic acid sequence or in an amino acid sequence, preferably in a nucleic acid sequence. The mutagen may be one or more physical agent (e.g., irradiation with ultraviolet radiation; ionizing radiation such as X-rays, gamma-rays, alpha particles; or radioactive decay), one or more chemical agent (e.g., reactive oxygen species (ROS) such as superoxide, hydroxyl radicals or hydrogen peroxide; reactive nitrogen species (RNS): intercalating agents such as ethidium bromide or 4′,6 diamidino 2 phenylindole; nucleoside analogs such as 5 azacytidine or 5 hydroxydeoxycytidine; metals such as arsenic, cadmium, chromium, or nickel; organic solvents; aromatic amines; aromatic hydrocarbons; alkylating agents; sodium azide; bromine; asbestos; deaminating agents; or mutagenic drugs), one or more nucleic acid molecule (e.g., base analog, transposon, or oligonucleotide), one or more peptide or protein (preferably an enzyme, more preferably a DNA and/or RNA binding enzyme, even more preferably a deaminase), one or more biological agent (e.g., an integrating virus, a retrovirus, an oncovirus, or a bacteria), or combinations thereof. Within the scope of the present invention, the term mutagen does not extend to evolutionary pressure, i.e., the conditions external to an organism and a genome that influence the selection of a mutation (e.g., reproductive benefit, survival, etc.).

“Mutated”, in connection with a sequence (e.g., an amino acid sequence or a nucleic acid sequence) means that the sequence is different than a reference sequence, such as a wild-type (WT) sequence. Typically, a mutated sequence comprises at least one of a substitution, an addition or a deletion of one or several residues by comparison to a reference sequence, such as a corresponding WT sequence.

“Nucleic acid (sequence)” and “nucleotide sequence” may be used interchangeably to refer to any molecule composed of, or comprising, monomeric nucleotides. A nucleic acid may be an oligonucleotide or a polynucleotide; it can be a DNA, an RNA, or a mix thereof. It can be chemically modified or artificial; e.g., it encompasses peptide nucleic acids (PNA), morpholinos and locked nucleic acids (LNA), as well as glycol nucleic acids (GNA) and threose nucleic acid (TNA). Each of these nucleic acids distinguish from naturally occurring DNA or RNA by changes in the backbone of the molecule. Also, phosphorothioate nucleotides may be used. Other deoxynucleotide analogs include, without limitation, methylphosphonates, phosphoramidates, phosphorodithioates, N3′P5′ phosphoramidates and oligoribonucleotide phosphorothioates and their 2′O-allyl analogs and 2′O-methylribonucleotide methylphosphonates which may be used in a nucleic acid of the disclosure.

“Polypeptide”, “peptide”, “protein” and “amino acid sequence” are used interchangeably to refer to a polymer of amino acid residues. Unless specified, a polymer of amino acid residues can be any length. The term also applies to amino acid polymers in which one or more amino acids are chemical analogues or modified derivatives of corresponding naturally-occurring amino acids.

“Sequence of interest” or “SoI”, sometimes also referred to as “gene of interest” (GoI) or “transgene (of interest)”, refers to any nucleic acid sequence encoding a product of interest. The product of interest may be a protein or a fragment thereof; in this case, the sequence of interest is said to be a “coding nucleic acid sequence”. However, the term also encompasses “non-coding nucleic acid sequences”, i.e., nucleic acid sequences that do not encode a protein or a fragment thereof. In some embodiments, the non-coding nucleic acid sequence expresses an “RNA gene” (or “non-coding RNA”, by opposition to a messenger RNA), such as, e.g., a transfer RNA, a ribosomal RNA, a small RNA, a long non-coding RNA, etc. In some alternative embodiments, the non-coding nucleic acid sequence is a regulatory element, such as, e.g., a promoter, an enhancer, a silencer, an insulator, an origin of replication, a terminator, a ribosome binding site, an internal ribosome entry site, a boundary element, a matrix attachment site, a locus control region, etc.

“Transfection” and any declension thereof refers to the introduction of one or several nucleic acid molecules (DNA and/or RNA) into one or more cells by non-viral means, whether in vitro or in vivo. Methods for transfection are well known in the art and include, e.g., lipofection and electroporation.

“Vector” as used herein, refer to any polynucleotide that can carry, e.g., a second polynucleotide of interest, and e.g., which can transfer gene sequences to target cells. Thus, the term includes cloning, and expression vehicles, as well as integrating vectors.

### DETAILED DESCRIPTION

The present invention relates to a method of synthetic evolution of a nucleic acid sequence of interest (SoI).

The method of the invention comprises the following steps, each of which will be described in more details herein:
(a) co-transfecting mammalian producer cells with:
   (i) a first nucleic acid molecule encoding a Retroviridae genome comprising at least the gag, pol and rev genes, and
   (ii) a second nucleic acid molecule encoding a recombinant expression cassette comprising, from 5′ to 3′:
      a 5′ long terminal repeat (5′ LTR),
      a packaging sequence,
      the SoI to be evolved, and
      a 3′ long terminal repeat (3′ LTR);
   (iii) optionally, a third nucleic acid molecule encoding a viral envelope glycoprotein;
(b) incubating the mammalian producer cells under conditions allowing for mutations of the SoI and production of Retroviridae vectors;
(c) harvesting the population of Retroviridae vectors produced at step (b);
(d) infecting reporter mammalian cells with the population of Retroviridae vectors produced at step (b), and optionally harvested at step (c);
(e) incubating the reporter mammalian cells under conditions allowing for SoI expression; and
(f) selecting one or several mutants of the SoI with a desired biological activity.
   As will be readily understood by a skilled artisan, these steps may be reiterated until a mutant of the SoI with the desired biological activity is obtained.

The method of the invention starts with the co-transfection of mammalian producer cells.

The mammalian producer cells may be human or non-human mammalian cells.

Non-limiting examples of non-human mammalian cells include simian cells, bovine cells, porcine cells, feline cells, canine cells, rabbit cells, hamster cells, rat cells, and mouse cells. Some specific non-limiting examples of non-human mammalian cells include Vero cells (from Cercopithecus sabaeus); BS-C-1 and CV-1 cells (from Cercopithecus aethiops); LLC-MK1, LLC-MK2 and FRhK-4 cells (from Macaca mulatta); MDBK cells (from Bos taurus); MDOK cells (from Ovis aries); CRFK cells (from Felis catus); MDCK cells (from Canis lupus familiaris); RK13 and LLC-RK1 cells (from Oryctolagus cuniculus); CHO cells (from Cricetulus griseus); BHK21-F cells (from Mesocricetus auratus); NRK and KNRK cells (from Ratus norvegicus); TCMK-1 and TKPTS cells (from Mus musculus).

In some preferred embodiment, the mammalian producer cells are human cells.

The human producer cells may be cells from an immortalized cell line, or they may be primary cells. As used herein, the term “primary cells” refers to cells freshly isolated from an organism, typically from a specific tissue. Preferably, a culture of primary cells comprises at most one cell type.

In some embodiments, the human producer cells are cells from an immortalized cell line. Non-limiting examples of immortalized human cell lines include 22Rv1, A204, A375, A549, AsPC-1, AU565, BxPC-3, Caco-2, DLD-1, ES-2, H929, HAP-1, HEK293, HEK293T, Hel92.1.7, HeLa, Hep-2, HS766T, IMR-32, Jurkat, K-562, MCF7, MIA PaCa-2, MM.1S, MOLT-4, NCI-H1299, NCI-H460, NCI-H647, NTH:OVCAR-3, Panc 08.13, PC-3, Ramos, RD, SK-N-DZ, SW1271, SW1990, SW620, T-47D, TF-1, THP-1, TOV-21G, and U-2 OS cell lines.

In some preferred embodiments, the human producer cells are selected from a group consisting of HEK293T cells, Jurkat cells and K-562 cells. In some preferred embodiments, the human producer cells are HEK293T cells. In some preferred embodiments, the human producer cells are Jurkat cells. In some preferred embodiments, the human producer cells are K-562 cells.

In some embodiments, the human producer cells are primary human cells. In some embodiments, the primary human cells are derived from a tissue from a live or deceased human donor.

According to the invention, the first nucleic acid molecule encodes a Retroviridae genome comprising at least the gag and pol genes.

The first nucleic acid molecule may be referred to as “packaging plasmid”.

In some embodiments, the Retroviridae genome further comprises the rev and/or tat genes. Alternatively, these accessory genes may be provided in a separate nucleic acid molecule.

Retroviruses are enveloped viruses from the Retroviridae family. They package two identical single-stranded ribonucleic acid (RNA) molecules of typically 7 to 10 kb in length, forming their genome. The genome of retroviruses typically comprises gag, pol and env genes flanked by two long terminal repeat (LTRs) sequences. Each of these genes encodes numerous peptides, which are initially expressed in the form of a single precursor polypeptide. The gag gene encodes the internal structure proteins (matrix, capsid and nucleocapsid); and the pol gene encodes retroviral enzymes reverse transcriptase, integrase and protease. The genome of retroviruses can further contain cis-acting elements, e.g., elements responsible for exporting out of the nucleus the unspliced viral genomic RNA which will be packaged, such as a Rev-response element (RRE) sequence. The 5′ and 3′ LTRs serve to promote transcription and also serve as a polyadenylation sequence of the viral RNAs. Sequences necessary for the initiation of reverse transcription of the genome and for the encapsidation of viral RNA in particles (psi [Ψ] packaging element) are typically adjacent to the 5′ LTR. If the sequences necessary for encapsidation are absent from the viral genome, the genomic RNA will not be actively packaged. The genome of more complex retroviruses may comprise additional genes encoding accessory and/or regulatory proteins such as src, sag, tax, vif, vpr, vpx, vpu, nef, tat, rev, tmx, tas and/or bet. For example, the HIV-1 genome contains 7 accessory genes: vif, vpr, vpx, vpu, nef, tat and rev.

The Retroviridae family is subdivided into two subfamilies: Orthoretrovirinae and Spumaretrovirinae. In some embodiments, the Retroviridae is (or is derived from) an Orthoretrovirinae or a Spumaretrovirinae. In some preferred embodiments, the Retroviridae is (or is derived from) an Orthoretrovirinae.

The Orthoretrovirinae subfamily is itself subdivided into six genera: Alpharetrovirus, Betaretrovirus, Deltaretrovirus, Epsilonretrovirus, Gammaretrovirus, and Lentivirus.

Exemplary species of Alpharetrovirus include, but are not limited to, avian sarcoma leukosis virus (ASLV), Rous sarcoma virus (RSV), and avian myeloblastosis virus (AMV).

Exemplary species of Betaretrovirus include, but are not limited to, mouse mammary tumor virus (MMTV), Jaagsiekte sheep retrovirus (JSRV), enzootic nasal tumor viruses (ENTV; including ENTV-1 and ENTV-2), simian retroviruses (SRV; including SRV-1 and SRV-2), and Mason-Pfizer monkey virus (M-PMV; formerly known as SRV-3).

Exemplary species of Deltaretrovirus include, but are not limited to, human T-lymphotropic viruses (HTLV; including HTLV-1, HTLV-2, HTLV-3 and HTLV-4), simian T-lymphotropic viruses (STLV; including STLV-1, STLV-2, STLV-3, and STLV-4), and bovine leukemia virus (BLV).

Exemplary species of Epsilonretrovirus include, but are not limited to, Walleye dermal sarcoma virus (WDSV), and Walleye epidermal hyperplasia viruses (WEHV; including WEHV-1 and WEHV-2).

Exemplary species of Gammaretrovirus include, but are not limited to, murine leukemia viruses (MLV), Abelson murine leukemia virus (AMLV), Friend virus (FV), feline leukemia virus (FeLV), koala retrovirus (KoRV), xenotropic murine leukemia virus-related virus (XMRV), chick syncytial virus (CSV), murine sarcoma viruses (MSV; including Finkel-Biskis-Jinkins murine sarcoma virus, Harvey murine sarcoma virus, Kirsten murine sarcoma virus and Moloney murine sarcoma virus), feline sarcoma viruses (FSV; including Gardner-Arnstein feline sarcoma virus, Hardy-Zuckerman feline sarcoma virus and Snyder-Theilen feline sarcoma virus), Gibbon ape leukemia virus (GaLV), guinea pig type-C oncovirus, porcine type-C oncovirus, reticuloendotheliosis virus, Trager duck spleen necrosis virus, viper retrovirus, and Woolly monkey sarcoma virus.

Exemplary species of Lentivirus include, but are not limited to, human immunodeficiency viruses (HIV; including HIV-1 and HIV-2), simian immunodeficiency viruses (SIV), feline immunodeficiency virus (FIV), bovine immunodeficiency virus (BIV), puma Lentivirus (PLV), equine infectious anemia virus (EIAV), caprine arthritis encephalitis virus (CAEV), Visna-maedi virus, and Jembrana disease virus.

In some embodiments, the Retroviridae is (or is derived from) an Alpharetrovirus, a Betaretrovirus, a Deltaretrovirus, an Epsilonretrovirus, a Gammaretrovirus or a Lentivirus.

In some preferred embodiments, the Retroviridae is (or is derived from) a Lentivirus. In some preferred embodiments, the Retroviridae is (or is derived from) a Lentivirus selected from the group comprising or consisting of human immunodeficiency viruses (HIV; including HIV-1 and HIV-2), simian immunodeficiency viruses (SIV), feline immunodeficiency virus (FIV), bovine immunodeficiency virus (BIV), puma Lentivirus (PLV), equine infectious anemia virus (EIAV), caprine arthritis encephalitis virus (CAEV), Visna-maedi virus (VMV), and Jembrana disease virus (JDV).

In some preferred embodiments, the Retroviridae is (or is derived from) a human immunodeficiency virus, such as HIV-1 and HIV-2; more preferably HIV-1.

In some embodiments, the Retroviridae genome comprises a pol gene coding for an error-prone reverse transcriptase/RNaseH mutant. As used herein, “error-prone” means that the reverse transcriptase/RNaseH mutant produces 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold or more mutations (i.e., nucleotide misincorporation) than a wild-type reverse transcriptase/RNaseH, over a fixed period of time. In other words, an error-prone reverse transcriptase/RNaseH mutant increases the rate of appearance of mutations in a nucleic acid molecule.

In some embodiments, the error-prone reverse transcriptase/RNaseH mutant comprises one or several mutations at position Y115, Q151, M184, M230 and/or Y501, said position number corresponding to the amino acid number of SEQ ID NO: 1; or at a positionally equivalent position in another reverse transcriptase/RNaseH, as can be determined by sequence and/or structural alignment.

SEQ ID NO: 1
PISPIETVPVKLKPGMDGPKVKQWPLTEEKIKALVEICTEMEKEGKISKI
GPENPYNTPVFAIKKKDSTKWRKLVDFRELNKRTQDFWEVQLGIPHPAGL
KQKKSVTVLDVGDAYFSVPLDKDFRKYTAFTIPSINNETPGIRYQYNVLP
QGWKGSPAIFQCSMTKILEPFRKQNPDIVIYQYMDDLYVGSDLEIGQHRT
KIEELRQHLLRWGFTTPDKKHQKEPPFLWMGYELHPDKWTVQPIVLPEKD
SWTVNDIQKLVGKLNWASQIYAGIKVRQLCKLLRGTKALTEVVPLTEEAE
LELAENREILKEPVHGVYYDPSKDLIAEIQKQGQGQWTYQIYQEPFKNLK
TGKYARMKGAHTNDVKQLTEAVQKIATESIVIWGKTPKFKLPIQKETWEA
WWTEYWQATWIPEWEFVNTPPLVKLWYQLEKEPIVGAETFYVDGAANRET
KLGKAGYVTNRGRQKVVTLTDTTNQKTELQAIYLALQDSGLEVNIVTDSQ
YALGIIQAQPDQSESELVNQIIEQLIKKEKVYLAWVPAHKGIGGNEQVDK
LVSAGIRKVLFLDGID

In some embodiments, the error-prone reverse transcriptase/RNaseH mutant comprises one or more mutations selected from the group consisting of Y115A, M184V, M184I, Q151M, M230I and Y501W, said position number corresponding to the amino acid number of SEQ ID NO: 1; or one or more positionally equivalent mutations in another reverse transcriptase/RNaseH, as can be determined by sequence and/or structural alignment.

In some embodiments, the error-prone reverse transcriptase/RNaseH mutant shares at least 75%, 80%, 85%, 90%, 95%, 96%, 97%, 98%, or 99% of sequence identity with SEQ ID NO: 1, while (i) retaining its reverse transcriptase activity and (ii) introducing more nucleotide misincorporations in a nucleic acid molecule than a wild-type reverse transcriptase/RNaseH (e.g., a wild-type reverse transcriptase/RNaseH with SEQ ID NO: 1).

In some embodiments, the error-prone reverse transcriptase/RNaseH mutant shares at least 75%, 80%, 85%, 90%, 95%, 96%, 97%, 98%, or 99% of sequence identity with SEQ ID NO: 1, while (i) retaining its reverse transcriptase activity and (ii) introducing more nucleotide misincorporations in a nucleic acid molecule than a wild-type reverse transcriptase/RNaseH.

In some embodiments, the error-prone reverse transcriptase/RNaseH mutant shares at least 75%, 80%, 85%, 90%, 95%, 96%, 97%, 98%, 99% or 100% of sequence identity with any of SEQ ID NOs: 2-7, while (i) retaining its reverse transcriptase activity and (ii) introducing more nucleotide misincorporations in a nucleic acid molecule than a wild-type reverse transcriptase/RNaseH.

SEQ ID NO: 2
PISPIETVPVKLKPGMDGPKVKQWPLTEEKIKALVEICTEMEKEGKISKI
GPENPYNTPVFAIKKKDSTKWRKLVDFRELNKRTQDFWEVQLGIPHPAGL
KQKKSVTVLDVGDAAFSVPLDKDFRKYTAFTIPSINNETPGIRYQYNVLP
QGWKGSPAIFQCSMTKILEPFRKQNPDIVIYQYMDDLYVGSDLEIGQHRT
KIEELRQHLLRWGFTTPDKKHQKEPPFLWMGYELHPDKWTVQPIVLPEKD
SWTVNDIQKLVGKLNWASQIYAGIKVRQLCKLLRGTKALTEVVPLTEEAE
LELAENREILKEPVHGVYYDPSKDLIAEIQKQGQGQWTYQIYQEPFKNLK
TGKYARMKGAHTNDVKQLTEAVQKIATESIVIWGKTPKFKLPIQKETWEA
WWTEYWQATWIPEWEFVNTPPLVKLWYQLEKEPIVGAETFYVDGAANRET
KLGKAGYVTNRGRQKVVTLTDTTNQKTELQAIYLALQDSGLEVNIVTDSQ
YALGIIQAQPDQSESELVNQIIEQLIKKEKVYLAWVPAHKGIGGNEQVDK
LVSAGIRKVLFLDGID
SEQ ID NO: 3
PISPIETVPVKLKPGMDGPKVKQWPLTEEKIKALVEICTEMEKEGKISKI
GPENPYNTPVFAIKKKDSTKWRKLVDFRELNKRTQDFWEVQLGIPHPAGL
KQKKSVTVLDVGDAYFSVPLDKDFRKYTAFTIPSINNETPGIRYQYNVLP
QGWKGSPAIFQCSMTKILEPFRKQNPDIVIYQYVDDLYVGSDLEIGQHRT
KIEELRQHLLRWGFTTPDKKHQKEPPFLWMGYELHPDKWTVQPIVLPEKD
SWTVNDIQKLVGKLNWASQIYAGIKVRQLCKLLRGTKALTEVVPLTEEAE
LELAENREILKEPVHGVYYDPSKDLIAEIQKQGQGQWTYQIYQEPFKNLK
TGKYARMKGAHTNDVKQLTEAVQKIATESIVIWGKTPKFKLPIQKETWEA
WWTEYWQATWIPEWEFVNTPPLVKLWYQLEKEPIVGAETFYVDGAANRET
KLGKAGYVTNRGRQKVVTLTDTTNQKTELQAIYLALQDSGLEVNIVTDSQ
YALGIIQAQPDQSESELVNQIIEQLIKKEKVYLAWVPAHKGIGGNEQVDK
LVSAGIRKVLFLDGID
SEQ ID NO: 4
PISPIETVPVKLKPGMDGPKVKQWPLTEEKIKALVEICTEMEKEGKISKI
GPENPYNTPVFAIKKKDSTKWRKLVDFRELNKRTQDFWEVQLGIPHPAGL
KQKKSVTVLDVGDAYFSVPLDKDFRKYTAFTIPSINNETPGIRYQYNVLP
QGWKGSPAIFQCSMTKILEPFRKQNPDIVIYQYIDDLYVGSDLEIGQHRT
KIEELRQHLLRWGFTTPDKKHQKEPPFLWMGYELHPDKWTVQPIVLPEKD
SWTVNDIQKLVGKLNWASQIYAGIKVRQLCKLLRGTKALTEVVPLTEEAE
LELAENREILKEPVHGVYYDPSKDLIAEIQKQGQGQWTYQIYQEPFKNLK
TGKYARMKGAHTNDVKQLTEAVQKIATESIVIWGKTPKFKLPIQKETWEA
WWTEYWQATWIPEWEFVNTPPLVKLWYQLEKEPIVGAETFYVDGAANRET
KLGKAGYVTNRGRQKVVTLTDTTNQKTELQAIYLALQDSGLEVNIVTDSQ
YALGIIQAQPDQSESELVNQIIEQLIKKEKVYLAWVPAHKGIGGNEQVDK
LVSAGIRKVLFLDGID
SEQ ID NO: 5
PISPIETVPVKLKPGMDGPKVKQWPLTEEKIKALVEICTEMEKEGKISKI
GPENPYNTPVFAIKKKDSTKWRKLVDFRELNKRTQDFWEVQLGIPHPAGL
KQKKSVTVLDVGDAYFSVPLDKDFRKYTAFTIPSINNETPGIRYQYNVLP
MGWKGSPAIFQCSMTKILEPFRKQNPDIVIYQYMDDLYVGSDLEIGQHRT
KIEELRQHLLRWGFTTPDKKHQKEPPFLWMGYELHPDKWTVQPIVLPEKD
SWTVNDIQKLVGKLNWASQIYAGIKVRQLCKLLRGTKALTEVVPLTEEAE
LELAENREILKEPVHGVYYDPSKDLIAEIQKQGQGQWTYQIYQEPFKNLK
TGKYARMKGAHTNDVKQLTEAVQKIATESIVIWGKTPKFKLPIQKETWEA
WWTEYWQATWIPEWEFVNTPPLVKLWYQLEKEPIVGAETFYVDGAANRET
KLGKAGYVTNRGRQKVVTLTDTTNQKTELQAIYLALQDSGLEVNIVTDSQ
YALGIIQAQPDQSESELVNQIIEQLIKKEKVYLAWVPAHKGIGGNEQVDK
LVSAGIRKVLFLDGID
SEQ ID NO: 6
PISPIETVPVKLKPGMDGPKVKQWPLTEEKIKALVEICTEMEKEGKISKI
GPENPYNTPVFAIKKKDSTKWRKLVDFRELNKRTQDFWEVQLGIPHPAGL
KQKKSVTVLDVGDAYFSVPLDKDFRKYTAFTIPSINNETPGIRYQYNVLP
QGWKGSPAIFQCSMTKILEPFRKQNPDIVIYQYMDDLYVGSDLEIGQHRT
KIEELRQHLLRWGFTTPDKKHQKEPPFLWIGYELHPDKWTVQPIVLPEKD
SWTVNDIQKLVGKLNWASQIYAGIKVRQLCKLLRGTKALTEVVPLTEEAE
LELAENREILKEPVHGVYYDPSKDLIAEIQKQGQGQWTYQIYQEPFKNLK
TGKYARMKGAHTNDVKQLTEAVQKIATESIVIWGKTPKFKLPIQKETWEA
WWTEYWQATWIPEWEFVNTPPLVKLWYQLEKEPIVGAETFYVDGAANRET
KLGKAGYVTNRGRQKVVTLTDTTNQKTELQAIYLALQDSGLEVNIVTDSQ
YALGIIQAQPDQSESELVNQIIEQLIKKEKVYLAWVPAHKGIGGNEQVDK
LVSAGIRKVLFLDGID
SEQ ID NO: 7
PISPIETVPVKLKPGMDGPKVKQWPLTEEKIKALVEICTEMEKEGKISKI
GPENPYNTPVFAIKKKDSTKWRKLVDFRELNKRTQDFWEVQLGIPHPAGL
KQKKSVTVLDVGDAYFSVPLDKDFRKYTAFTIPSINNETPGIRYQYNVLP
QGWKGSPAIFQCSMTKILEPFRKQNPDIVIYQYMDDLYVGSDLEIGQHRT
KIEELRQHLLRWGFTTPDKKHQKEPPFLWMGYELHPDKWTVQPIVLPEKD
SWTVNDIQKLVGKLNWASQIYAGIKVRQLCKLLRGTKALTEVVPLTEEAE
LELAENREILKEPVHGVYYDPSKDLIAEIQKQGQGQWTYQIYQEPFKNLK
TGKYARMKGAHTNDVKQLTEAVQKIATESIVIWGKTPKFKLPIQKETWEA
WWTEYWQATWIPEWEFVNTPPLVKLWYQLEKEPIVGAETFYVDGAANRET
KLGKAGYVTNRGRQKVVTTDTTNQKTELQAIYLALQDSGLEVNIVTDSQL
WALGIIQAQPDQSESELVNQIIEQLIKKEKVYLAWVPAHKGIGGNEQVDK
LVSAGIRKVLFLDGID

According to the invention, the second nucleic acid molecule encodes a recombinant expression cassette comprising, from 5′ to 3′:
a 5′ long terminal repeat (5′ LTR),
a packaging sequence,
the nucleic acid sequence of interest (SoI) to be evolved, and
a 3′ long terminal repeat (3′ LTR).

The second nucleic acid molecule may be referred to as “transfer plasmid”.

In some embodiments, the sequence of interest (SoI) is a coding nucleic acid sequence or a non-coding nucleic acid sequence.

The term “sequence of interest” or “SoI”, sometimes also referred to as “transgene (of interest)”, refers to any nucleic acid sequence encoding a product of interest. The product of interest may be a protein or a fragment thereof; in this case, the sequence of interest is said to be a “coding nucleic acid sequence”. However, the term also encompasses “non-coding nucleic acid sequences”, i.e., nucleic acid sequences that do not encode a protein or a fragment thereof. In some embodiments, the non-coding nucleic acid sequence expresses an “RNA gene” (or “non-coding RNA”, by opposition to a messenger RNA), such as, e.g., a transfer RNA, a ribosomal RNA, a small RNA, a long non-coding RNA, etc. In some alternative embodiments, the non-coding nucleic acid sequence is a regulatory element, such as, e.g., a promoter, an enhancer, a silencer, an insulator, an origin of replication, a terminator, a ribosome binding site, an internal ribosome entry site, a boundary element, a matrix attachment site, a locus control region, etc.

In some embodiments, the SoI is a coding nucleic acid sequence, encoding a peptide or protein, including, without limitation, an enzyme, a genome editor, a nuclease, a recombinase, a transposase, a transcription factor, a growth factor, a trophic factor, a hormone, a cytokine, an antibody, an antigen, a receptor, an immune regulator, a differentiation factor, a suicide protein, a cell-cycle modifying protein, an anti-proliferative protein, an angiogenic factor, an anti-angiogenic factor, a neurotransmitter, and a reporter, including any precursor thereof, as well as fusion proteins. In this case, the SoI may typically be (or be derived from) an mRNA, a cDNA, a gDNA, a synthetic nucleic acid, or any combinations thereof.

In some embodiments, the SoI is a non-coding nucleic acid sequence.

In some embodiments, the SoI is a regulatory element including, but not limited to, a promoter, an enhancer, a silencer, an insulator, an origin of replication, a terminator, a ribosome binding site, an internal ribosome entry site, a boundary element, a matrix attachment site, and a locus control region.

In some embodiments, the SoI expresses a non-coding RNA including, but are not limited to, a transfer RNA (tRNA), a ribosomal RNA (rRNA), a small nuclear RNA (snRNA), a small nucleolar RNA (snoRNA), a SmY RNA, a small Cajal body-specific RNA (scaRNA), a guide RNA (gRNA), a Y RNA, a telomerase RNA component (TERC), a spliced leader RNA (SL RNA), a catalytic RNA (i.e., ribozymes; such as, e.g., ribonuclease P, ribonuclease MRP, and the like), an antisense RNA (aRNA), a cis-natural antisense transcript (cis-NAT), a CRISPR RNA (crRNA), a long non-coding RNA (lncRNA), a microRNA (miRNA), a piwi-interacting RNA (piRNA), a small interfering RNA (siRNA), a short hairpin RNA (shRNA), a trans-acting siRNA (tasiRNA), a repeat-associated siRNA (rasiRNA), a 7SK RNA (7SK), an enhancer RNA (eRNA), and an RNA aptamer.

In some embodiments, the SoI is flanked by two long terminal repeats.

“Long-terminal repeats”, or “LTR”, are sequences of several hundred base pairs long. In RNA viruses, their genome is flanked by LTRs (a 5′ LTR and a 3′ LTR), typically having identical sequences. LTR are segmented into three regions, named U3, R, and U5 (in this order from 5′ to 3′).

In some embodiments, the Retroviridae vectors produced from the co-transfection of mammalian producer cells are not “self-inactivating”. Hence, according to this embodiment, the 5′ LTR and 3′ LTR each comprise a U3 site (containing an enhancer and promoter sequence that drive viral transcription), an R site (encoding the 5′-capping and polyA sequences), and a U5 site. In particular, the U3 site of the 5′ LTR is not substituted with an exogenous enhancer/promoter, in particular, is not substituted with a truncated cytomegalovirus (CMV) immediate early (IE) enhancer/TATA promoter; and/or the U3 site of the 3′ LTR is not deleted.

In some embodiments, the second nucleic acid molecule may further comprise one or several elements, in particular one or several of a packaging sequence, a Rev-response element (RRE) sequence, and a post-transcriptional regulation element sequence.

By “packaging sequence”, it is referred to a stem-loop structured cis-acting nucleic acid sequence, which regulates the process of packaging inside a viral capsid. This packaging sequence may be referred in the art to as “packaging signal” denoted “y”, or “encapsidation signal” denoted “E”. Packaging sequences may be of viral origin (i.e., wild-type viral packaging sequences) or may be synthetic.

Examples of packaging sequences include, but are not limited to, the psi (Ψ) packaging signal of HIV or SIV; the core encapsidation signal from Gammaretrovirus; the epsilon (ε) encapsidation signal from HBV, and the encapsidation signal from BLV.

The “Rev-response element” is a highly structured RNA segment interacting with the Rev protein, allowing the viral genome to be exported to the cytoplasm for downstream processing, including virion packaging. Rev-response elements are typically characteristic of lentiviruses, but other RNA viruses of Group VI comprise similar systems, such as the Rem-response element in Betaretroviruses, the Rex-response element in Deltaretroviruses, or the constitutive transport element (CTE). These are also encompassed when mentioning Rev-response element herein, even if not explicitly cited.

The “post-transcriptional regulation element” is a cis-acting RNA sequence that can increase the accumulation of cytoplasmic mRNA by promoting mRNA exportation from the nucleus to the cytoplasm, enhancing 3′-end processing and stability.

Examples of post-transcriptional regulatory element include, but are not limited to, the post-transcriptional regulatory element of Woodchuck hepatitis virus (WPRE) and the post-transcriptional regulatory element of hepatitis B virus (HPRE).

In some embodiments, the second nucleic acid molecule may thus comprise, from 5′ to 3′:
a 5′ LTR, comprising a U3, R and U5 sites,
a packaging sequence,
optionally, a Rev-response element,
a nucleic acid SoI,
optionally, a post-transcriptional regulatory element, and
a 3′ LTR, comprising a U3, R and U5 sites.

According to the invention, the third nucleic acid molecule encodes a viral envelope glycoprotein.

The third nucleic acid molecule may be referred to as “envelope plasmid”.

The envelope glycoprotein may be from (or derived from) an enveloped virus, possibly from a Retroviridae or from any other enveloped virus, such as, e.g., from a Rhabdoviridae. It may also be a cellular glycoprotein or a synthetic glycoprotein.

In some embodiments, the third nucleic acid molecule encodes an envelope glycoprotein from Indiana vesiculovirus (formerly known as vesicular stomatitis virus or VSV).

In some embodiments, the mammalian producer cells are not co-transfected with the third nucleic acid molecule. In some embodiments, the mammalian producer cells are co-transfected with the third nucleic acid molecule.

Means and method to transfect cells with nucleic acid molecules are well-known to the skilled artisan.

Non-limiting examples of such means and methods include lipofection, cationic polymer-based transfection (e.g., using polyethyleneimine), calcium phosphate-based transfection, fugene-based transfection, dendrimer-based transfection, electroporation, microinjection, gene gun, impalefection, hydrostatic pressure, continuous infusion, sonication, and magnetofection.

In some embodiments, transfection is performed by lipofection. Means of performing lipofection are known in the art and comprise using a lipofection reagent such as, e.g., Lipofectamine™. In some embodiments, transfection is performed by electroporation. In some embodiments, transfection is performed by sonication. In some embodiments, transfection is performed using nanoparticles (e.g., polymeric nanoparticles such as JetPEI). In some embodiments, transfection is performed by microinjection.

Once the mammalian producer cells were co-transfected, they are incubated under conditions allowing for mutations of the SoI and production of Retroviridae vectors (or Retroviridae particles, comprising at least one copy of the SoI possibly with one or several mutations as compared to the SoI of the second nucleic acid molecule).

In some embodiments, the mammalian producer cells are cultured in a culture medium suitable for their growth. It shall be understood that the skilled artisan readily knows which culture medium is suitable depending on the cell type. Non-limiting examples of culture medium include Minimum Essential Medium (MEM), Eagle's minimal essential medium (EMEM), Dulbecco's Modified Eagle's Medium (DMEM), RPMI 1640 medium, Iscove's Modified Dulbecco's Medium (IMDM), Roswell Park Memorial Institute medium (RPMI), Ham's tissue culture medium, William's Medium, and F-12 medium.

In some embodiments, the culture medium is supplemented with nutrients. Non-limiting examples of nutrients include sugars, amino acids, proteins, vitamins, fatty acids, lipids, and combinations thereof. An example of suitable sugar is glucose. An example of suitable amino acid is glutamate or a derivative thereof, e.g., glutamine.

In some embodiments, the culture medium is further supplemented with growth factors. The skilled artisan knows how to select suitable growth factors depending on the cell type. In some embodiments, the growth factors are provided in a serum. Non-limiting examples of serum include bovine serum, fetal bovine serum (FBS), goat serum, horse serum, sheep serum, rabbit serum and chicken serum.

In some embodiments, the culture medium is further supplemented with agents inhibiting the growth of, and/or eliminating, microorganisms (e.g., bacteria, fungi, mycoplasmas and the like), typically an antibiotic agent or an antifungal agent, or combinations thereof. Example of suitable antibiotic agents include penicillin and streptomycin.

In some embodiments, the culture medium comprises a pH buffer, preferably a pH buffer suitable for atmospheric condition comprising 5% CO₂.

In some embodiments, the culture medium is suitable for culturing HEK293 cells, preferably HEK293T cells. In some embodiments, the culture medium is Dulbecco's modified Eagle medium. In some embodiments, the culture medium is Dulbecco's modified Eagle medium supplemented with high glucose, 10% fetal bovine serum, 2 mM glutamine, 100 U penicillin and 0.1 mg/mL streptomycin, as exemplified in Example 1 below.

In some embodiments, the culture medium is suitable for culturing Jurkat cells and/or K-562 cells. In some embodiments, the culture medium is RPMI 1640 medium. In some embodiments, the culture medium is RPMI 1640 medium supplemented with 10% FBS, 1% penicillin-streptomycin and 1% GlutaMAX, as exemplified in Example 1 below.

In some embodiments, the mammalian producer cells are incubated at a temperature ranging from about 30° C. to about 44° C., preferably from about 35° C. to about 39° C., more preferably from about 36° C. to about 38° C., even more preferably at about 37° C.

In some embodiments, the mammalian producer cells are incubated under a proportion of CO₂ in air ranging from about 1% to about 9%, preferably from about 2% to about 8%, more preferably from about 3% to about 7%, even more preferably from about 4% to about 6%, even more preferably at about 5%.

According to the invention, the SoI is mutated in the Retroviridae vectors during step (b).

These mutations may be selected from a group consisting of substitutions (including transversions and transitions), deletions, insertions, inversions and any combinations thereof.

In some embodiments, the mammalian producer cells are incubated at step (b) in the presence of at least one mutagen.

Although Retroviridae vectors have a basal mutagenesis rate allowing for mutations of the SoI, the mammalian producer cells may be incubated in the presence of a mutagen to generate more mutations and/or specific types of mutations. As used herein, the term “mutagen” refers to a compound or process that results in the introduction of mutations in the SoI.

Examples of mutagens include irradiation (e.g., ultraviolet, X-rays, gamma-rays, alpha particles and the like), DNA intercalating agents (e.g., ethidium bromide, 4′,6-diamidino-2-phenylindole and the like), reactive oxygen species (e.g., hydrogen peroxide, hydroxyl, superoxide and the like) and mutagenic drugs. Mutagens are known from the art, and the examples of mutagens provided herein are not to be interpreted imitatively.

In some embodiments, the mutagen is one or more agent selected from the group comprising or consisting of physical agents, chemical agents, nucleic acid molecules, peptides or proteins, biological agents, and any combination thereof. In some embodiments, the mutagen is one or more agent selected from the group comprising or consisting of chemical agents, nucleic acid molecules, peptides or proteins, and any combination thereof.

In some embodiments, the mutagen is selected from the group comprising or consisting of physical agents, chemical agents, nucleic acid molecules, peptides or proteins, and biological agents. In some embodiments, the mutagen is one or more agent selected from the group comprising or consisting of chemical agents, nucleic acid molecules, and peptides or proteins.

In some embodiments, the mutagen is a combination of one or more chemical agent, one or more nucleic acid molecule, and one or more peptide or protein. In some embodiments, the mutagen is a combination of one or more chemical agent, and one or more nucleic acid molecule. In some embodiments, the mutagen is a combination of one or more chemical agent, and one or more peptide or protein. In some embodiments, the mutagen is a combination of one or more nucleic acid molecule, and one or more peptide or protein. In some embodiments, the mutagen is a combination of a chemical agent, a nucleic acid molecule, and a peptide or protein. In some embodiments, the mutagen is a combination of a chemical agent, and a nucleic acid molecule. In some embodiments, the mutagen is a combination of a chemical agent, and a peptide or protein. In some embodiments, the mutagen is a combination of a nucleic acid molecule, and a peptide or protein.

In one embodiment, the mutagen is one or more chemical agent. In one embodiment, the mutagen is a chemical agent.

In some embodiment, the chemical agent is selected from the group comprising or consisting of reactive oxygen species (ROS) such as superoxide, hydroxyl radicals or hydrogen peroxide, reactive nitrogen species (RNS), intercalating agents such as ethidium bromide or 4′,6 diamidino 2 phenylindole, nucleoside analogs such as 5 azacytidine or 5 hydroxydeoxycytidine, metals such as arsenic, cadmium, chromium, or nickel, organic solvents, aromatic amines, aromatic hydrocarbons, alkylating agents, sodium azide, bromine, asbestos, deaminating agents, mutagenic drugs, and combinations thereof. In some embodiment, the chemical agent is selected from the group comprising or consisting of ROS, intercalating agents, and mutagenic drugs. In some embodiment, the chemical agent is a ROS. In some embodiment, the chemical agent is an intercalating agent.

In some preferred embodiments, the chemical agent is a mutagenic drug. In some preferred embodiments, the mutagen is a mutagenic drug.

Non-limiting examples of mutagenic drugs include nucleoside analogs, aromatic hydrocarbons, aromatic amines, azides, alkylating agents and deaminating agents.

In some embodiments, the mutagenic drug is a nucleoside analog.

In some embodiments, the nucleoside analog is selected from the group consisting of 5-hydroxydeoxycytidine (5-OH-dC), 5-azacytidine (5-aza-C), 1-β-D-ribofuranosyl-1-H-1,2,4-triazole-3-carboxamide (a.k.a. ribavirin), 5-fluorouracil, 6-fluoro-3-hydroxy-pirazine-2-carboxamide (a.k.a. favipiravir), azidothymidine, 2′,3′-dideoxy-3′-thiacytidine (a.k.a. (+)-lamivudine), 2′-3′-didehydro-2′-3′-dideoxythymidine (a.k.a stavudine), 3′-azido-3′-deoxythymidine (a.k.a zidovudine), 5-aza-2′-deoxycytidine (a.k.a. decitabine), 2′-deoxy-5,6-dihydro-5-azacytidine (a.k.a KP-1212), 2′-3′-dideoxycytidine (ddC), and {(1S,4R)-4-[2-amino-6-(cyclopropylamino)-9H-purin-9-yl]-2-cyclopenten-1-yl}methanol (a.k.a. abacavir).

In some embodiments, the nucleoside analog is selected from the group consisting of 5-hydroxydeoxycytidine (5-OH-dC) and 5-azacytidine (5-aza-C). In some embodiments, the nucleoside analog is 5-hydroxydeoxycytidine (5-OH-dC). In some embodiments, the nucleoside analog is 5-azacytidine (5-aza-C).

In some embodiments, the mammalian producer cells are contacted with the nucleoside analog at a dose ranging from about 100 nM to about 10 mM, from about 100 nM to about 5 mM, from about 100 nM to about 1 mM, from about 100 nM to about 500 μM, from about 100 nM to about 100 μM, from about 100 nM to about 50 μM, 1 μM to about 10 mM, from about 1 μM to about 5 mM, from about 1 μM to about 1 mM, from about 1 μM to about 500 μM, from about 1 μM to about 100 μM, from about 1 μM to about 50 μM, 50 μM to about 10 mM, from about 50 μM to about 5 mM, from about 50 μM to about 1 mM, from about 50 μM to about 500 μM, from about 50 μM to about 100 μM.

In some embodiments, the mammalian producer cells are contacted with the nucleoside analog at a dose of about 100 nM, 200 nM, 300 nM, 400 nM, 500 nM, 600 nM, 700 nM, 800 nM, 900 nM, 1 μM, 10 μM, 50 μM, 100 μM, 200 μM, 300 μM, 400 μM, 500 μM, 600 μM, 700 μM, 800 μM, 900 μM, 1 mM, or 10 mM.

In some preferred embodiments, the mammalian producer cells are contacted with the nucleoside analog at a dose ranging from about 50 μM to about 1 mM, such as a dose of about 50 μM, 100 μM, 200 μM, 300 μM, 400 μM, 500 μM, 600 μM, 700 μM, 800 PM, 900 μM or 1 mM.

In one embodiment, the mutagen is one or more peptide or protein. In one embodiment, the mutagen is a peptide or protein.

In some embodiments, the peptide or protein is a DNA binding protein and/or a RNA binding protein.

In a preferred embodiment, the peptide or protein is an enzyme. In some embodiments, the enzyme is selected from the group comprising or consisting of a deaminase, a transposase, a nuclease, a nickase, a ligase, and a restriction enzyme. In some embodiments, the enzyme is a deaminase, a transposase or a nuclease. In some embodiments, the enzyme is a deaminase or a transposase.

In some embodiments, the enzyme is a deaminase, or a fragment thereof. In some embodiments, the mutagen is a deaminase. “Deaminases” are enzymes which catalyze the removal of an amino group from a molecule (i.e., a deamination). Deaminases can target nucleic acids, in particular their nucleobases. Deamination of adenosine results in the formation of hypoxanthine, which then selectively base pairs with cytosine instead of thymine, leading to an A-to-G substitution. Deamination of cytosine results in the formation of uracil, which then selectively base pairs with adenine instead of guanosine, leading to a C-to-T substitution. Deamination of guanosine results in the formation of xanthine; however, xanthine still base pairs with cytosine.

In some embodiments, the deaminase is a single-stranded or double-stranded RNA-specific deaminase.

In some embodiments, the deaminase is an adenosine deaminase or a cytidine deaminase.

In some embodiments, the deaminase is a single-stranded or double-stranded RNA-specific adenosine deaminase or a single-stranded or double-stranded RNA-specific cytidine deaminase.

Examples of suitable RNA-specific deaminases include, without limitation, proteins from the “double-stranded RNA-specific adenosine deaminase” or “ADAR” family (including ADAR1; and ADARB1 [a.k.a ADAR2]), AMP deaminase 1, proteins from the “apolipoprotein B mRNA editing enzymes” or “APOBEC” superfamily (including C→U-editing enzyme APOBEC-1; probable C→U-editing enzyme APOBEC-2; and C→U-editing enzyme APOBEC-4), and cytidine deaminase (or “CDA”).

In some preferred embodiments, the deaminase is a protein from the ADAR family, a catalytically active domain thereof, or a mutant thereof which retains its deaminase activity or has an improved deaminase activity as compared to the corresponding wild-type deaminase.

In some preferred embodiments, the deaminase is ADARB1.

“ADARB1”, also named “double-stranded RNA-specific editase 1” or “ADAR2”, is an enzyme encoded in humans by the ADARB1 gene. An exemplary amino acid sequence of the canonical human ADARB1 is SEQ ID NO: 8. A catalytically active domain of ADARB1 comprises or consists of amino acid residues 316-741 or 370-741 or 316-737 or 370-737 of SEQ ID NO: 8. A mutant of ADARB1 which has improved deaminase activity is a E528Q and/or T375G mutant (SEQ ID NO: 8 numbering).

SEQ ID NO: 8
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQ
TGPVHAPLFVMSVEVNGQVFEGSGPTKKKAKLHAAEKALRSFVQFPNASE
AHLAMGRTLSVNTDFTSDQADFPDTLFNGFETPDKAEPPFYVGSNGDDSF
SSSGDLSLSASPVPASLAQPPLPVLPPFPPPSGKNPVMILNELRPGLKYD
FLSESGESHAKSFVMSVVVDGQFFEGSGRNKKLAKARAAQSALAAIFNLH
LDQTPSRQPIPSEGLQLHLPQVLADAVSRLVLGKFGDLTDNFSSPHARRK
VLAGVVMTTGTDVKDAKVISVSTGTKCINGEYMSDRGLALNDCHAEIISR
RSLLRFLYTQLELYLNNKDDQKRSIFQKSERGGFRLKENVQFHLYISTSP
CGDARIFSPHEPILEGSRSYTQAGVQWCNHGSLQPRPPGLLSDPSTSTFQ
GAGTTEPADRHPNRKARGQLRTKIESGEGTIPVRSNASIQTWDGVLQGER
LLTMSCSDKIARWNVVGIQGSLLSIFVEPIYFSSIILGSLYHGDHLSRAM
YQRISNIEDLPPLYTLNKPLLSGISNAEARQPGKAPNFSVNWTVGDSAIE
VINATTGKDELGRASRLCKHALYCRWMRVHGKVPSHLLRSKITKPNVYHE
SKLAAKEYQAAKARLFTAFIKAGLGAWVEKPTEQDQFSLTP

Isoform 9 of human ADARB1 with SEQ ID NO: 9 is also suitable for use in the present invention. A catalytically active domain of ADARB1 comprises or consists of amino acid residues 365-750 or 419-750 or 365-746 or 419-746 of SEQ ID NO: 9. A mutant of ADARB1 which has improved deaminase activity is a E537Q and/or T424G mutant (SEQ ID NO: 9 numbering).

SEQ ID NO: 9
MASSTTGPLHMGTFFSIMGRRYKRRRKKRSERKDRNSLRQSRNPQKYFAM
DIEDEENMSSSSTDVKENRNLDNVSPKDGSTPGPGEGSQLSNGGGGGPGR
KRPLEEGSNGHSKYRLKKRRKTPGPVLPKNALMQLNEIKPGLQYTLLSQT
GPVHAPLFVMSVEVNGQVFEGSGPTKKKAKLHAAEKALRSFVQFPNASEA
HLAMGRTLSVNTDFTSDQADFPDTLFNGFETPDKAEPPFYVGSNGDDSFS
SSGDLSLSASPVPASLAQPPLPVLPPFPPPSGKNPVMILNELRPGLKYDF
LSESGESHAKSFVMSVVVDGQFFEGSGRNKKLAKARAAQSALAAIFNLHL
DQTPSRQPIPSEGLQLHLPQVLADAVSRLVLGKFGDLTDNFSSPHARRKV
LAGVVMTTGTDVKDAKVISVSTGTKCINGEYMSDRGLALNDCHAEIISRR
SLLRFLYTQLELYLNNKDDQKRSIFQKSERGGFRLKENVQFHLYISTSPC
GDARIFSPHEPILEEPADRHPNRKARGQLRTKIESGEGTIPVRSNASIQT
WDGVLQGERLLTMSCSDKIARWNVVGIQGSLLSIFVEPIYFSSIILGSLY
HGDHLSRAMYQRISNIEDLPPLYTLNKPLLSGISNAEARQPGKAPNFSVN
WTVGDSAIEVINATTGKDELGRASRLCKHALYCRWMRVHGKVPSHLLRSK
ITKPNVYHESKLAAKEYQAAKARLFTAFIKAGLGAWVEKPTEQDQFSLTP

In some embodiments, the deaminase, a catalytically active domain thereof, or a mutant thereof which retains its deaminase activity or has an improved deaminase activity as compared to the corresponding wild-type deaminase, is expressed in the mammalian producer cells. Means and method for recombinantly expressing a protein in a cell are well-known to the skilled artisan. In some embodiments, the mammalian producer cells are stably expressing the deaminase.

In another embodiment, the enzyme is a transposase.

In some embodiments, the mutagen is one or more nucleic acid molecules. In one embodiment, the mutagen is a nucleic acid molecule.

In some embodiments, the nucleic acid molecule is single stranded. In some embodiments, the nucleic acid molecule is double stranded. In some embodiments, the nucleic acid molecule is a DNA molecule. In some embodiments, the nucleic acid molecule is a RNA molecule. In some embodiments, the nucleic acid molecule has a length from 5 nucleotides to 500 nucleotides, from 5 nucleotides to 100 nucleotides, or from 5 nucleotides to 50 nucleotides.

In some embodiments, the nucleic acid molecule is selected from a library of nucleic acid molecules. In one embodiment, the nucleic acid molecules of the library have a mutagenic effect.

In some embodiments, the nucleic acid molecule comprises or encodes a premature stop codon. In some embodiments, the nucleic acid molecule comprises or encodes a coding sequence. In some embodiments, the nucleic acid molecule comprises or encodes a regulatory sequence.

In some embodiments, the nucleic acid molecule comprises or encodes a fragment of the SoI. In some embodiments, the fragment of the SoI comprises 1, 2, 3, 4, 5 or more mutations.

In some embodiments, the mutagen is selected from the group comprising or consisting of irradiation, DNA intercalating agents, reactive oxygen species, nucleic acids, and mutagenic drugs. In some embodiments, the mutagen is selected from the group comprising or consisting of irradiation, DNA intercalating agents, reactive oxygen species, and mutagenic drugs. In some embodiments, the mutagenic drug is a nucleoside analog. In some embodiments, the mutagen is further complemented with a deaminase.

It shall be understood that two or more mutagens described herein may be used in combination, e.g., a nucleoside analog and a deaminase.

In some embodiments, the mammalian producer cells are incubated at step (b) in the presence of at least one deaminase and at least one nucleoside analog. In some embodiments, the mammalian producer cells are incubated at step (b) in the presence of a deaminase and a nucleoside analog. In some embodiments, the mammalian producer cells are incubated at step (b) in the presence of a deaminase and 5-hydroxydeoxycytidine (5-OH-dC). In some embodiments, the mammalian producer cells are incubated at step (b) in the presence of a deaminase and 5-azacytidine (5-aza-C).

In some embodiments, the mammalian producer cells are incubated at step (b) in the presence of a deaminase and a nucleic acid analog selected from a library as defined hereinabove.

In some embodiments, the mammalian producer cells are incubated at step (b) in the presence of a nucleoside analog and a nucleic acid analog selected from a library as defined hereinabove.

In some embodiments, the mammalian producer cells are incubated at step (b) in the presence of a deaminase, a nucleoside analog, and a nucleic acid analog selected from a library as defined hereinabove.

Mutagens, in particular deaminases, can be recruited to a specific region of the SoI, allowing targeted mutagenesis of a portion of the SoI. For instance, the portion of the SoI may be a region of the sequence encoding a functional domain, for instance, a domain of a protein with enzymatic or binding activity.

In some embodiments, the deaminase may be recruited to a specific region of the SoI using a guide RNA.

Guide RNAs have been developed to target deaminases to specific sequences.

Typically, the guide RNA comprises two regions: a first region binding to the deaminase, and a second region being at least partially complementary to a specific sub-sequence of the SoI. In that respect, see, e.g., Fukuda et al., 2017 (Sci Rep. 7:41478), Doherty et al., 2021 (J Am Chem Soc. 143(18):6865-6876), Nose et al., 2021 (Nucleic Acid Ther. 31(1):58-67), and Katrekar et al., 2022 (Nat Biotechnol. 40(6):938-945).

Hence, in some embodiments, the mammalian producer cells are transfected or otherwise contacted with a guide RNA recruiting the deaminase to a specific sub-sequence of the SoI.

Alternatively, the deaminase may be recruited to a specific region of the SoI by fusing it to an RNA targeting CRISPR effector. In some embodiments, the deaminase is thus fused to an RNA targeting CRISPR effector, optionally with a linker or spacer.

Examples of RNA targeting CRISPR effectors include, without limitation, Cas13 (including Cas13a, Cas13b, Cas13c and Cas13d), Cas9 and Cas12a. In some preferred embodiments, the RNA targeting CRISPR effector is catalytically inactive (some referred to as “dead” in the art, e.g., “dead Cas13” or “dCas13”), i.e., it is devoid of RNAse activity.

In a preferred embodiment, the RNA targeting CRISPR effector is Cas13b, preferably a catalytically inactive Cas13b (dCas13b).

Exemplary amino acid sequences of dCas13b include SEQ ID NO: 10 (dCas13b from anaerobic digester metagenome 15706; dAdmCas13b), SEQ ID NO: 11 (dCas13b from Eubacterium siraeum DSM15702; dEsCas13b), SEQ ID NO: 12 (dCas13b from Lachnospiraceae bacterium; dLbaCas13b), SEQ ID NO: 13 (dCas13b from Leptotrichia wadei; dLwaCas13b), SEQ ID NO: 14 (dCas13b from Porphyromonas gulae; dPguCas13b), SEQ ID NO: 15 (dCas13b from Prevotella sp. P5-125; dPspCas13b), SEQ ID NO: 16 (dCas13b from Ruminoccocus flavefaciens XPD3002; dRanCas13b), and SEQ ID NO: 17 (dCas13b from Ruminococcus flavefaciens; dRfxCas13b).

SEQ ID NO: 10
MNNKRKTKAKAAGLKSVFFDQKQAVLTTFAKGNNSQIEKKVVNSEVKDLRQPPAFDL
ELKEKTFYISGKNNINTSRENPLASASLPLSKRQRIRAERIKRAREENRPYHNVKRVGED
DLRAKADLEKHYFGKEYSDNLKIQIIYNILDINKIISPYINDIVYSMNNLARNDEYIDGKI
DVIGSLSSTTDYSSFMSPNKDLEKEKKFSFHRENYKKFVEASKPYMRYYGKVFIRDVKK
SKLSTGKGEKIEVMYRSDEEIFTIFQILSYVAQSIMANDIGNKSSILAIEKYPARFVGFLSD
LLKTKTNDVNRMFIDNNSQTNFWVLFSIFGLQDHTSGADKICRNFYDFVIKADSKNLGF
SLKKIRELMLDLPNANMLRDHQFDTVRSKFYTLLDFIIYQHYLEEKSRIDNMVEKLRMT
LKEEEKEVLYAAEAKIVWNAIGAKVINKLVPMMNGDALKEIKRKNRDRKLPQSVIATV
QVNSDANVFSGLIYFLTLFLDGKEINEMVSNLITKFENIDSLLHVDREIYKSDEKDLDLEI
EKLALFFKGVVRPNAKTDTGAGEISKSFSIFQSAERIIEELKFIKNVTRMDNEIFPSEGVFL
DAANVLGVRGDDFDFSNEFVGDDLHSDANKKIINKINGTKEDRNLRNFIINNVVKSRRF
QYIARHMNTHYVKQLANNETLNRFVLNKMGDAKIINRYYESISGNTPNIEVRSQIDYLV
KRLRSFSFEDLNDVKQKVRPGTNESIEKEKKKALVGLCLTIQYLVYKNLVNINARYTTA
FYCLERDSKLKGFGVDVWRDFESYTALTNHFIKEGYLPVRKAEILRANLKHLDCEDGF
KYYANQVTALNAIRVAYKYINEIKSVHSYFALYHYIMQRHLYDSLQAKAKDSSGFVID
ALKKSFEHKIYSKDLLHVLHSPFGYNTARYKNLSIEALFDKNESRPEVNPLSTND
SEQ ID NO: 11
MGKKIHARDLREQRKTDRTEKFADQNKKREAERAVPKKDAAVSVKSVSSVSSKKDNV
TKSMAKAAGVKSVFAVGNTVYMTSFGRGNDAVLEQKIVDTSHEPLNIDDPAYQLNVV
TMNGYSVTGHRGETVSAVTDNPLRRFNGRKKDEPEQSVPTDMLCLKPTLEKKFFGKEF
DDNIHIQLIYNILDIEKILAVYSTNAIYALNNMSADENIENSDFFMKRTTDETFDDFEKKK
ESTNSREKADFDAFEKFIGNYRLAYFADAFYVNKKNPKGKAKNVLREDKELYSVLTLI
GKLAHWCVASEEGRAEFWLYKLDELKDDFKNVLDVVYNRPVEEINNRFIENNKVNIQI
LGSVYKNTDIAELVRSYYEFLITKKYKNMGFSIKKLRESMLEGKGYADKEYDSVRNKL
YQMTDFILYTGYINEDSDRADDLVNTLRSSLKEDDKTTVYCKEADYLWKKYRESIREV
ADALDGDNIKKLSKSNIEIQEDKLRKCFISYADSVSEFTKLIYLLTRFLSGKEINDLVTTLI
NKFDNIRSFLEIMDELGLDRTFTAEYSFFEGSTKYLAELVELNSFVKSCSFDINAKRTMY
RDALDILGIESDKTEEDIEKMIDNILQIDANGDKKLKKNNGLRNFIASNVIDSNRFKYLV
RYGNPKKIRETAKCKPAVRFVLNEIPDAQIERYYEACCPKNTALCSANKRREKLADMIA
EIKFENFSDAGNYQKANVTSRTSEAEIKRKNQAIIRLYLTVMYIMLKNLVNVNARYVIA
FHCVERDTKLYAESGLEVGNIEKNKTNLTMAVMGVKLENGIIKTEFDKSFAENAANRY
LRNARWYKLILDNLKKSERAVVNEFANTVCALNAIRNININIKEIKEVENYFALYHYLIQ
KHLENRFADKKVERDTGDFISKLEEHKTYCKDFVKAYCTPFGYNLVRYKNLTIDGLFD
KNYPGKDDSDEQK
SEQ ID NO: 12
MKISKVREENRGAKLTVNAKTAVVSENRSQEGILYNDPSRYGKSRKNDEDRDRYIESR
LKSSGKLYRIFNEDKNKRETDELQWFLSEIVKKINRRNGLVLSDMLSVDDRAFEKAFEK
YAELSYTNRRNKVSGSPAFETCGVDAATAERLKGIISETNFINRIKNNIDNKVSEDIIDRII
AKYLKKSLCRERVKRGLKKLLMNAFDLPYSDPDIDVQRDFIDYVLEDFYHVRAKSQVS
RSIKNMNMPVQPEGDGKFAITVSKGGTESGNKRSAEKEAFKKFLSDYASLDERVRDDM
LRRMRRLVVLYFYGSDDSKLSDVNEKFDVWEDHAARRVDNREFIKLPLENKLANGKT
DKDAERIRKNTVKELYRNQNIGCYRQAVKAVEEDNNGRYFDDKMLNMFFIHRIEYGV
EKIYANLKQVTEFKARTGYLSEKIWKDLINYISIKYIAMGKAVYNYAMDELNASDKKEI
ELGKISEEYLSGISSFDYELIKAEEMLQRETAVYVAFAARHLSSQTVELDSENSDFLLLK
PKGTMDKNDKNKLASNNILNFLKDKETLRDTILQYFGGHSLWTDFPFDKYLAGGKDD
VDFLTDLKDVIYSMANDSFAYATENHNNGKWNKELISAMFEHETERMTVVMKDKFYS
NNLPMFYKNDDLKKLLIDLYKDNVERASQVPSFNKVFVRKNFPALVRDKDNLGIELDL
KADADKGENELKFYNALYYMFKEIYYNAFLNDKNVRERFITKATKVADNYDRNKERN
LKDRIKSAGSDEKKKLREQLQNYIAENDFGQRIKNIVQVNPDYTLAQICQLIMTEYNQQ
NNGCMQKKSAARKDINKDSYQHYKMLLLVNLRKAFLEFIKENYAFVLKPYKHDLCDK
ADFVPDFAKYVKPYAGLISRVAGSSELQKWYIVSRFLSPAQANHMLGFLHSYKQYVW
DIYRRASETGTEINHSIAEDKIAGVDITDVDAVIDLSVKLCGTISSEISDYFKDDEVYAEYI
SSYLDFEYDGGNYKDSLNRFCNSDAVNDQKVALYYDGEHPKLNRNIILSKLYGERRFL
EKITDRVSRSDIVEYYKLKKETSQYQTKGIFDSEDEQKNIKKFQEMKNIVEFRDLMDYS
EIADELQGQLINWIYLRERDLMNFQLGYHYACLNNDSNKQATYVTLDYQGKKNRKIN
GAILYQICAMYINGLPLYYVDKDSSEWTVSDGKESTGAKIGEFYRYAKSFENTSDCYAS
GLEIFENISEHDNITELANYIEAFRYYSSFDRSFLGIYSEVFDRFFTYDLKYRKNVPTILYN
ILLQHFVNVRFEFVSGKKMIGIDKKDRKIAKEKECARITIREKNGVYSEQFTYKLKNGT
VYVDARDKRYLQSIIRLLFYPEKVNMDEMIEVKEKKKPSDNNTGKGYSKRDRQQDRK
EYDKYKEKKKKEGNFLSGMGGNINWDEINAQLKN
SEQ ID NO: 13
KVTKVDGISHKKYIEEGKLVKSTSEENRTSERLSELLSIRLDIYIKNPDNASEEENRIRRE
NLKKFFSNKVLHLKDSVLYLKNRKEKNAVQDKNYSEEDISEYDLKNKNSFSVLKKILL
NEDVNSEELEIFRKDVEAKLNKINSLKYSFEENKANYQKINENNVEKVGGKSKRNIIYD
YYRESAKRNDYINNVQEAFDKLYKKEDIEKLFFLIENSKKHEKYKIREYYHKIIGRKND
KENFAKIIYEEIQNVNNIKELIEKIPDMSELKKSQVFYKYYLDKEELNDKNIKYAFCHFV
EIEMSQLLKNYVYKRLSNISNDKIKRIFEYQNLKKLIENKLLNKLDTYVRNCGKYNYYL
QVGEIATSDFIARNRQNEAFLRNIIGVSSVAYFSLRNILETENENDITGRMRGKTVKNNK
GEEKYVSGEVDKIYNENKQNEVKENLKMFYSYDFNMDNKNEIEDFFANIDEAISSIAHG
IVAFNLELEGKDIFAFKNIAPSEISKKMFQNEINEKKLKLKIFKQLNSANVFNYYEKDVII
KYLKNTKFNFVNKNIPFVPSFTKLYNKIEDLRNTLKFFWSVPKDKEEKDAQIYLLKNIY
YGEFLNKFVKNSKVFFKITNEVIKINKQRNQKTGHYKYQKFENIEKTVPVEYLAIIQSRE
MINNQDKEEKNTYIDFIQQIFLKGFIDYLNKNNLKYIESNNNNDNNDIFSKIKIKKDNKE
KYDKILKNYEKHNRNKEIPHEINEFVREIKLGKILKYTENLNMFYLILKLLNHKELTNLK
GSLEKYQSANKEETFSDELELINLLNLDNNRVTEDFELEANEIGKFLDFNENKIKDRKEL
KKFDTNKIYFDGENIIKHRAFYNIKKYGMLNLLEKIADKAKYKISLKELKEYSNKKNEIE
KNYTMQQNLHRKYARPKKDEKFNDEDYKEYEKAIGNIQKYTHLKNKVEFNELNLLQG
LLLKILHRLVGYTSIWERDLRFRLKGEFPENHYIEEIFNFDNSKNVKYKSGQIVEKYINF
YKELYKDNVEKRSIYSDKKVKKLKQEKKDLYIANYIAAFNYIPHAEISLLEVLENLRKL
LSYDRKLKNAIMKSIVDILKEYGFVATFKIGADKKIEIQTLESEKIVHLKNLKKKKLMTD
RNSEELCELVKVMFEYKALEG
SEQ ID NO: 14
TEQSERPYNGTYYTLEDKHFWAAFLNLARHNAYITLTHIDRQLAYSKADITNDQDVLS
FKALWKNFDNDLERKSRLRSLILKHESFLEGAAYGKKLFESKSSGNKSSKNKELTKKEK
EELQANALSLDNLKSILFDFLQKLKDFRNYYSAYRHSGSSELPLFDGNMLQRLYNVFD
VSVQRVKIDHEHNDEVDPHYHFNHLVRKGKKDRYGHNDNPSFKHHFVDGEGMVTEA
GLLFFVSLFLEKRDAIWMQKKIRGFKGGTETYQQMTNEVFCRSRISLPKLKLESLRMDD
WMLLDMLNELVRCPKPLYDRLREDDRACFRVPVDILPDEDDTDGGGEDPFKNTLVRH
QDRFPYFALRYFDLKKVFTSLRFHIDLGTYHFAIYKKMIGEQPEDRHLTRNLYGFGRIQ
DFAEEHRPEEWKRLVRDLDYFETGDKPYISQTSPHYHIEKGKIGLRFMPEGQHLWPSPE
VGTTRTGRSKYAQDKRLTAEAFLSVHELMPMMFYYFLLREKYSEEVSAERVQGRIKRV
IEDVYAVYDAFARDEINTRDELDACLADKGIRRGHLPRQMIAILSQEHKDMEEKIRKKL
QEMMADTDHRLDMLDRQTDRKIRIGRKNAGLPKSGVIADWLVRDMMRFQPVAKDAS
GKPLNNSKANSTEYRMLQRALALEGGEKERLTPYFRQMNLTGGNNPHPFLHETRWES
HTNILSFYRSYLRARKAFLERIGRSDRVENRPFLLLKEPKTDRQTLVAGWKGEFHLPRGI
FTEAVRDCLIEMGHDEVASYKEVGFMAKAVPLYFERACEDRVQPFYDSPFNVGNSLKP
KKGRFLSKEERAEEWERGKERFRDLEAWSYSAARRIEDAFAGIEYASPGNKKKIEQLLR
DLSLWEAFESKLKVRADRINLAKLKKEILEAQEHPYHDFKSWQKFERELRLVKNQDIIT
WMMCRDLMEENKVEGLDTGTLYLKDIRPNVQEQGSLNVLNRVKPMRLPVVVYRADS
RGHVHKEEAPLATVYIEERDTKLLKQGNFKSFVKDRRLNGLFSFVDTGGLAMEQYPIS
KLRVEYELAKYQTARVCVFELTLRLEESLLTRYPHLPDESFREMLESWSDPLLAKWPEL
HGKVRLLIAVRNAFSANQYPMYDEAVESSIRKYDPSSPDAIEERMGLNIAHRLSEEVKQ
AKETVERIIQA
SEQ ID NO: 15
NIPALVENQKKYFGTYSVMAMLNAQTVLDHIQKVADIEGEQNENNENLWFHPVMSHL
YNAKNGYDKQPEKTMFIIERLQSYFPFLKIMAENQREYSNGKYKQNRVEVNSNDIFEVL
KRAFGVLKMYRDLTNAYKTYEEKLNDGCEFLTSTEQPLSGMINNYYTVALRNMNERY
GYKTEDLAFIQDKRFKFVKDAYGKKKSQVNTGFFLSLQDYNGDTQKKLHLSGVGIALL
ICLFLDKQYINIFLSRLPIFSSYNAQSEERRIIIRSFGINSIKLPKDRIHSEKSNKSVAMDML
NEVKRCPDELFTTLSAEKQSRFRIISDDHNEVLMKRSSDRFVPLLLQYIDYGKLFDHIRF
HVNMGKLRYLLKADKTCIDGQTRVRVIEQPLNGFGRLEEAETMRKQENGTFGNSGIRI
RDFENMKRDDANPANYPYIVDTYTHYILENNKVEMFINDKEDSAPLLPVIEDDRYVVK
TIPSCRMSTLEIPAMAFHMFLFGSKKTEKLIVDVHNRYKRLFQAMQKEEVTAENIASFGI
AESDLPQKILDLISGNAHGKDVDAFIRLTVDDMLTDTERRIKRFKDDRKSIRSADNKMG
KRGFKQISTGKLADFLAKDIVLFQPSVNDGENKITGLNYRIMQSAIAVYDSGDDYEAKQ
QFKLMFEKARLIGKGTTEPHPFLYKVFARSIPANAVEFYERYLIERKFYLTGLSNEIKKG
NRVDVPFIRRDQNKWKTPAMKTLGRIYSEDLPVELPRQMFDNEIKSHLKSLPQMEGIDF
NNANVTYLIAEYMKRVLDDDFQTFYQWNRNYRYMDMLKGEYDRKGSLQHCFTSVEE
REGLWKERASRTERYRKQASNKIRSNRQMRNASSEEIETILDKRLSNSRNEYQKSEKVI
RRYRVQDALLFLLAKKTLTELADFDGERFKLKEIMPDAEKGILSEIMPMSFTFEKGGKK
YTITSEGMKLKNYGDFFVLASDKRIGNLLELVGSDIVSKEDIMEEFNKYDQCRPEISSIVF
NLEKWAFDTYPELSARVDREEKVDFKSILKILLNNKNINKEQSDILRKIRNAFDANNYP
DKGVVEIKALPEIAMSIKKAFGEYAIMK
SEQ ID NO: 16
EKPLLPNVYTLKHKFFWGAFLNIARHNAFITICHINEQLGLKTPSNDDKIVDVVCETWN
NILNNDHDLLKKSQLTELILKHFPFLTAMCYHPPKKEGKKKGHQKEQQKEKESEAQSQ
AEALNPSKLIEALEILVNQLHSLRNYYSAYKHKKPDAEKDIFKHLYKAFDASLRMVKE
DYKAHFTVNLTRDFAHLNRKGKNKQDNPDFNRYRFEKDGFFTESGLLFFTNLFLDKRD
AYWMLKKVSGFKASHKQREKMTTEVFCRSRILLPKLRLESRYDHNQMLLDMLSELSR
CPKLLYEKLSEENKKHFQVEADGFLDEIEEEQNPFKDTLIRHQDRFPYFALRYLDLNESF
KSIRFQVDLGTYHYCIYDKKIGDEQEKRHLTRTLLSFGRLQDFTEINRPQEWKALTKDL
DYKETSNQPFISKTTPHYHITDNKIGFRLGTSKELYPSLEIKDGANRIAKYPYNSGFVAH
AFISVHELLPLMFYQHLTGKSEDLLKETVRHIQRIYKDFEEERINTIEDLEKANQGRLPL
GAFPKQMLGLLQNKQPDLSEKAKIKIEKLIAETKLLSHRLNTKLKSSPKLGKRREKLIKT
GVLADWLVKDFMRFQPVAYDAQNQPIKSSKANSTEFWFIRRALALYGGEKNRLEGYF
KQTNLIGNTNPHPFLNKFNWKACRNLVDFYQQYLEQREKFLEAIKNQPWEPYQYCLLL
KIPKENRKNLVKGWEQGGISLPRGLFTEAIRETLSEDLMLSKPIRKEIKKHGRVGFISRAI
TLYFKEKYQDKHQSFYNLSYKLEAKAPLLKREEHYEYWQQNKPQSPTESQRLELHTSD
RWKDYLLYKRWQHLEKKLRLYRNQDVMLWLMTLELTKNHFKELNLNYHQLKLENL
AVNVQEADAKLNPLNQTLPMVLPVKVYPATAFGEVQYHKTPIRTVYIREEHTKALKM
GNFKALVKDRRLNGLFSFIKEENDTQKHPISQLRLRRELEIYQSLRVDAFKETLSLEEKL
LNKHTSLSSLENEFRALLEEWKKEYAASSMVTDEHIAFIASVRNAFCANQYPFYKEALH
APIPLFTVAQPTTEEKDGLGIAEALLKVLREYCEIVKSQI
SEQ ID NO: 17
IEKKKSFAKGMGVKSTLVSGSKVYMTTFAEGSDARLEKIVEGDSIRSVNEGEAFSAEM
ADKNAGYKIGNAKFSHPKGYAVVANNPLYTGPVQQDMLGLKETLEKRYFGESADGN
DNICIQVIHNILDIEKILAEYITNAAYAVNNISGLDKDIIGFGKFSTVYTYDEFKDPEHHR
AAFNNNDKLINAIKAQYDEFDNFLDNPRLGYFGQAFFSKEGRNYIINYGNECYDILALL
SGLAHWVVANNEEESRISRTWLYNLDKNLDNEYISTLNYLYDRITNELTNSFSKNSAAN
VNYIAETLGINPAEFAEQYFRFSIMKEQKNLGFNITKLREVMLDRKDMSEIRKNHKVFD
SIRTKVYTMMDFVIYRYYIEEDAKVAAANKSLPDNEKSLSEKDIFVINLRGSFNDDQKD
ALYYDEANRIWRKLENIMHNIKEFRGNKTREYKKKDAPRLPRILPAGRDVSAFSKLMY
ALTMFLDGKEINDLLTTLINKFDNIQSFLKVMPLIGVNAKFVEEYAFFKDSAKIADELRL
IKSFARMGEPIADARRAMYIDAIRILGTNLSYDELKALADTFSLDENGNKLKKGKHGM
RNFIINNVISNKRFHYLIRYGDPAHLHEIAKNEAVVKFVLGRIADIQKKQGQNGKNQIDR
YYETCIGKDKGKSVSEKVDALTKIITGMNYDQFDKKRSVIEDTGRENAEREKFKKIISLY
LTVIYHILKNIVNINARYVIGFHCVERDAQLYKEKGYDINLKKLEEKGFSSVTKLCAGID
ETAPDKRKDVEKEMAERAKESIDSLESANPKLYANYIKYSDEKKAEEFTRQINREKAKT
ALNAYLRNTKWNVIIREDLLRIDNKTCTLFANKAVALEVARYVHAYINDIAEVNSYFQ
LYHYIMQRIIMNERYEKSSGKVSEYFDAVNDEKKYNDRLLKLLCVPFGYCIPRFKNLSI
EALFDRNEAAKFDKEKKKVSGNS

These RNA targeting CRISPR effectors are RNA-guided, meaning that they require a guide RNA to be recruited to a specific sequence. Similarly to guide RNAs recruiting deaminases, guide RNAs recruiting RNA targeting CRISPR effectors typically comprise a first region binding to the CRISPR effector and a second region being at least partially complementary to a specific sub-sequence of the SoI. In this case, the two regions of guide RNAs have been coined in the art “trans-activating crRNA” or “tracrRNA” for the first region, and “CRISPR RNA” or “crRNA” for the second region. As is well known in the art, the tracrRNA and the crRNA can be fused together to form a “single guide RNA” or can be provided separately as a “2-part guide RNA”. In the latter case, the 3′-end sequence of the crRNA is complementary to the 5′-end sequence of the tracrRNA to allow hybridization and complex formation.

Hence, in some embodiments, a fusion protein comprising (i) the deaminase, a catalytically active domain thereof, or a mutant thereof which retains its deaminase activity or has an improved deaminase activity as compared to the corresponding wild-type deaminase as described above, and (ii) the RNA targeting CRISPR effector as described above, is expressed in the mammalian producer cells. Means and method for recombinantly expressing a protein in a cell are well-known to the skilled artisan. In some embodiments, the mammalian producer cells are stably expressing the fusion protein.

According to the latter embodiment, the mammalian producer cells are further transfected with or otherwise contacted with a guide RNA, with a crRNA targeting a specific sub-sequence of the SoI.

In some embodiments, the guide RNA comprises a crRNA that is at least partially complementary to a sub-sequence of the SoI By “partially complementary”, it is meant that the sequence of the crRNA and the sequence of the sub-sequence of the SoI are at least 50%, 60%, 70%, 80%, 90%, 95% or more complementary with each other. It shall however be understood that, regardless of the percentage of complementarity between the sequence of the crRNA and the sequence of the sub-sequence, they should remain annealable under physiological conditions.

Exemplarily, the team of Feng Zhang has developed “REPAIR” for “RNA Editing for Programmable A to I Replacement” (Cox et al., 2017. Science. 358(6366):1019-1027), “RESCUE” for “RNA Editing for Specific C to U Exchange” (Abudayyeh et al., 2019. Science. 365(6451):382-386), and “CURE” for “C-to-U RNA editing” (Huang et al., 2020. EMBO J. 39(22):e104741).

All three systems use a catalytically inactivated Cas13 protein (dCas13) fused to a deaminase domain (of ADAR2 for the first two, and of APOBEC3A for the last one), recruited to a specific nucleic acid sequence using a guide RNA. These systems are suitable for use in the present invention.

An exemplary fusion protein used in the “REPAIR” (v.1) system comprises or consists of SEQ ID NO: 18. This fusion protein is for instance expressible in mammalian producer cells using the “pC0047-CMV-dPspCas13b-ADAR1DD(E1008Q)” plasmid from Feng Zhang's laboratory (publicly available at Addgene, plasmid ref. #103863).

SEQ ID NO: 18
MNIPALVENQKKYFGTYSVMAMLNAQTVLDHIQKVADIEGEQNENNENLW
FHPVMSHLYNAKNGYDKQPEKTMFIIERLQSYFPFLKIMAENQREYSNGK
YKQNRVEVNSNDIFEVLKRAFGVLKMYRDLTNAYKTYEEKLNDGCEFLTS
TEQPLSGMINNYYTVALRNMNERYGYKTEDLAFIQDKRFKFVKDAYGKKK
SQVNTGFFLSLQDYNGDTQKKLHLSGVGIALLICLFLDKQYINIFLSRLP
IFSSYNAQSEERRIIIRSFGINSIKLPKDRIHSEKSNKSVAMDMLNEVKR
CPDELFTTLSAEKQSRFRIISDDHNEVLMKRSSDRFVPLLLQYIDYGKLF
DHIRFHVNMGKLRYLLKADKTCIDGQTRVRVIEQPLNGFGRLEEAETMRK
QENGTFGNSGIRIRDFENMKRDDANPANYPYIVDTYTHYILENNKVEMFI
NDKEDSAPLLPVIEDDRYVVKTIPSCRMSTLEIPAMAFHMFLFGSKKTEK
LIVDVHNRYKRLFQAMQKEEVTAENIASFGIAESDLPQKILDLISGNAHG
KDVDAFIRLTVDDMLTDTERRIKRFKDDRKSIRSADNKMGKRGFKQISTG
KLADFLAKDIVLFQPSVNDGENKITGLNYRIMQSAIAVYDSGDDYEAKQQ
FKLMFEKARLIGKGTTEPHPFLYKVFARSIPANAVEFYERYLIERKFYLT
GLSNEIKKGNRVDVPFIRRDQNKWKTPAMKTLGRIYSEDLPVELPRQMFD
NEIKSHLKSLPQMEGIDFNNANVTYLIAEYMKRVLDDDFQTFYQWNRNYR
YMDMLKGEYDRKGSLQHCFTSVEEREGLWKERASRTERYRKQASNKIRSN
RQMRNASSEEIETILDKRLSNSRNEYQKSEKVIRRYRVQDALLFLLAKKT
LTELADFDGEREKLKEIMPDAEKGILSEIMPMSFTFEKGGKKYTITSEGM
KLKNYGDFFVLASDKRIGNLLELVGSDIVSKEDIMEEFNKYDQCRPEISS
IVFNLEKWAFDTYPELSARVDREEKVDFKSILKILLNNKNINKEQSDILR
KIRNAFDANNYPDKGVVEIKALPEIAMSIKKAFGEYAIMKGSLQLPPLER
LTLGSQLHLPQVLADAVSRLVLGKFGDLTDNFSSPHARRKVLAGVVMTTG
TDVKDAKVISVSTGTKCINGEYMSDRGLALNDCHAEIISRRSLLRFLYTQ
LELYLNNKDDQKRSIFQKSERGGFRLKENVQFHLYISTSPCGDARIFSPH
EPILEEPADRHPNRKARGQLRTKIESGQGTIPVRSNASIQTWDGVLQGER
LLTMSCSDKIARWNVVGIQGSLLSIFVEPIYFSSIILGSLYHGDHLSRAM
YQRISNIEDLPPLYTLNKPLLSGISNAEARQPGKAPNFSVNWTVGDSAIE
VINATTGKDELGRASRLCKHALYCRWMRVHGKVPSHLLRSKITKPNVYHE
SKLAAKEYQAAKARLFTAFIKAGLGAWVEKPTEQDQFSLT

Another exemplary fusion protein used in the “REPAIR” (v.2) system comprises or consists of SEQ ID NO: 19. This fusion protein is for instance expressible in mammalian producer cells using the “pC0054-CMV-dPspCas13b-longlinker-ADAR2DD(E488Q/T375G)” plasmid from Feng Zhang's laboratory (publicly available at Addgene, plasmid ref. #103870).

SEQ ID NO: 19
MNIPALVENQKKYFGTYSVMAMLNAQTVLDHIQKVADIEGEQNENNENLW
FHPVMSHLYNAKNGYDKQPEKTMFIIERLQSYFPFLKIMAENQREYSNGK
YKQNRVEVNSNDIFEVLKRAFGVLKMYRDLTNAYKTYEEKLNDGCEFLTS
TEQPLSGMINNYYTVALRNMNERYGYKTEDLAFIQDKRFKFVKDAYGKKK
SQVNTGFFLSLQDYNGDTQKKLHLSGVGIALLICLFLDKQYINIFLSRLP
IFSSYNAQSEERRIIIRSFGINSIKLPKDRIHSEKSNKSVAMDMLNEVKR
CPDELFTTLSAEKQSRFRIISDDHNEVLMKRSSDRFVPLLLQYIDYGKLF
DHIRFHVNMGKLRYLLKADKTCIDGQTRVRVIEQPLNGFGRLEEAETMRK
QENGTFGNSGIRIRDFENMKRDDANPANYPYIVDTYTHYILENNKVEMFI
NDKEDSAPLLPVIEDDRYVVKTIPSCRMSTLEIPAMAFHMFLFGSKKTEK
LIVDVHNRYKRLFQAMQKEEVTAENIASFGIAESDLPQKILDLISGNAHG
KDVDAFIRLTVDDMLTDTERRIKRFKDDRKSIRSADNKMGKRGFKQISTG
KLADFLAKDIVLFQPSVNDGENKITGLNYRIMQSAIAVYDSGDDYEAKQQ
FKLMFEKARLIGKGTTEPHPFLYKVFARSIPANAVEFYERYLIERKFYLT
GLSNEIKKGNRVDVPFIRRDQNKWKTPAMKTLGRIYSEDLPVELPRQMFD
NEIKSHLKSLPQMEGIDFNNANVTYLIAEYMKRVLDDDFQTFYQWNRNYR
YMDMLKGEYDRKGSLQHCFTSVEEREGLWKERASRTERYRKQASNKIRSN
RQMRNASSEEIETILDKRLSNSRNEYQKSEKVIRRYRVQDALLFLLAKKT
LTELADFDGERFKLKEIMPDAEKGILSEIMPMSFTFEKGGKKYTITSEGM
KLKNYGDFFVLASDKRIGNLLELVGSDIVSKEDIMEEFNKYDQCRPEISS
IVFNLEKWAFDTYPELSARVDREEKVDFKSILKILLNNKNINKEQSDILR
KIRNAFDANNYPDKGVVEIKALPEIAMSIKKAFGEYAIMKGSLQLPPLER
LTLGSGGGGSQLHLPQVLADAVSRLVLGKFGDLTDNFSSPHARRKVLAGV
VMTTGTDVKDAKVISVSTGGKCINGEYMSDRGLALNDCHAEIISRRSLLR
FLYTQLELYLNNKDDQKRSIFQKSERGGFRLKENVQFHLYISTSPCGDAR
IFSPHEPILEEPADRHPNRKARGQLRTKIESGQGTIPVRSNASIQTWDGV
LQGERLLTMSCSDKIARWNVVGIQGSLLSIFVEPIYFSSIILGSLYHGDH
LSRAMYQRISNIEDLPPLYTLNKPLLSGISNAEARQPGKAPNFSVNWTVG
DSAIEVINATTGKDELGRASRLCKHALYCRWMRVHGKVPSHLLRSKITKP
NVYHESKLAAKEYQAAKARLFTAFIKAGLGAWVEKSTEQDQFSLT

An exemplary fusion protein used in the “RESCUE” system comprises or consists of SEQ ID NO: 20 or SEQ ID NO: 21. These fusion proteins are for instance expressible in mammalian producer cells using the “pC0078 RESCUE” plasmid from Feng Zhang's laboratory (publicly available at Addgene, plasmid ref. #130661) or the “pC0079 RESCUE-S” plasmid from Feng Zhang's laboratory (publicly available at Addgene, plasmid ref. #130662).

SEQ ID NO: 20
MEKPLLPNVYTLKHKFFWGAFLNIARHNAFITICHINEQLGLKTPSNDDK
IVDVVCETWNNILNNDHDLLKKSQLTELILKHFPFLTAMCYHPPKKEGKK
KGHQKEQQKEKESEAQSQAEALNPSKLIEALEILVNQLHSLANYYSAYKH
KKPDAEKDIFKHLYKAFDASLRMVKEDYKAHFTVNLTRDFAHLNRKGKNK
QDNPDFNRYRFEKDGFFTESGLLFFTNLFLDKRDAYWMLKKVSGFKASHK
QREKMTTEVFCRSRILLPKLRLESRYDHNQMLLDMLSELSRCPKLLYEKL
SEENKKHFQVEADGFLDEIEEEQNPFKDTLIRHQDRFPYFALRYLDLNES
FKSIRFQVDLGTYHYCIYDKKIGDEQEKRHLTRTLLSFGRLQDFTEINRP
QEWKALTKDLDYKETSNQPFISKTTPHYHITDNKIGFRLGTSKELYPSLE
IKDGANRIAKYPYNSGFVAHAFISVHELLPLMFYQHLTGKSEDLLKETVR
HIQRIYKDFEEERINTIEDLEKANQGRLPLGAFPKQMLGLLQNKQPDLSE
KAKIKIEKLIAETKLLSHRLNTKLKSSPKLGKRREKLIKTGVLADWLVKD
FMRFQPVAYDAQNQPIKSSKANSTEFWFIRRALALYGGEKNRLEGYFKQT
NLIGNTNPHPFLNKFNWKACRNLVDFYQQYLEQREKFLEAIKNQPWEPYQ
YCLLLKIPKENRKNLVKGWEQGGISLPRGLFTEAIRETLSEDLMLSKPIR
KEIKKHGRVGFISRAITLYFKEKYQDKHQSFYNLSYKLEAKAPLLKREEH
YEYWQQNKPQSPTESQRLELHTSDRWKDYLLYKRWQHLEKKLRLYRNQDV
MLWLMTLELTKNHFKELNLNYHQLKLENLAVNVQEADAKLNPLNQTLPMV
LPVKVYPATAFGEVQYHKTPIRTVYIREEHTKALKMGNFKALVKDRRLNG
LFSFIKEENDTQKHPISQLRLRRELEIYQSLRVDAFKETLSLEEKLLNKH
TSLSSLENEFRALLEEWKKEYAASSMVTDEHIAFIASVANAFCANQYPFY
KEALHAPIPLFTVAQPTTEEKDGLGIAEALLKVLREYCEIVKSQIGSSLQ
KKLEELELGSQLHLPQVLADAVSRLVIGKFGDLTDNFSSPHARRIGLAGV
VMTTGTDVKDAKVICVSTGSKCINGEYLSDRGLALNDCHAEIVSRRSLLR
FLYTQLELYLNNEDDQKRSIFQKSERGGFRLKENIQFHLYISTSPCGDAR
IFSPHEAILEEPADRHPNRKARGQLRTKIEAGQGTIPVRNNASIQTWDGV
LQGERLLTMSCSDKIARWNVVGIQGSLLSIFVEPIYFSSIILGSLYHGDH
LSRAMYQRISNIEDLPPLYTLNKPLLTGISNAEARQPGKAPIFSVNWTVG
DSAIEVINATTGKGELGRASRLCKHALYCRWMRVHGKVPSHLLRSKITKP
NVYHETKLAAKEYQAAKARLFTAFIKAGLGAWVEKPTEQDQFSLT
SEQ ID NO: 21
MEKPLLPNVYTLKHKFFWGAFLNIARHNAFITICHINEQLGLKTPSNDDK
IVDVVCETWNNILNNDHDLLKKSQLTELILKHFPFLTAMCYHPPKKEGKK
KGHQKEQQKEKESEAQSQAEALNPSKLIEALEILVNQLHSLANYYSAYKH
KKPDAEKDIFKHLYKAFDASLRMVKEDYKAHFTVNLTRDFAHLNRKGKNK
QDNPDFNRYRFEKDGFFTESGLLFFTNLFLDKRDAYWMLKKVSGFKASHK
QREKMTTEVFCRSRILLPKLRLESRYDHNQMLLDMLSELSRCPKLLYEKL
SEENKKHFQVEADGFLDEIEEEQNPFKDTLIRHQDRFPYFALRYLDLNES
FKSIRFQVDLGTYHYCIYDKKIGDEQEKRHLTRTLLSFGRLQDFTEINRP
QEWKALTKDLDYKETSNQPFISKTTPHYHITDNKIGFRLGTSKELYPSLE
IKDGANRIAKYPYNSGFVAHAFISVHELLPLMFYQHLTGKSEDLLKETVR
HIQRIYKDFEEERINTIEDLEKANQGRLPLGAFPKQMLGLLQNKQPDLSE
KAKIKIEKLIAETKLLSHRLNTKLKSSPKLGKRREKLIKTGVLADWLVKD
FMRFQPVAYDAQNQPIKSSKANSTEFWFIRRALALYGGEKNRLEGYFKQT
NLIGNTNPHPFLNKFNWKACRNLVDFYQQYLEQREKFLEAIKNQPWEPYQ
YCLLLKIPKENRKNLVKGWEQGGISLPRGLFTEAIRETLSEDLMLSKPIR
KEIKKHGRVGFISRAITLYFKEKYQDKHQSFYNLSYKLEAKAPLLKREEH
YEYWQQNKPQSPTESQRLELHTSDRWKDYLLYKRWQHLEKKLRLYRNQDV
MLWLMTLELTKNHFKELNLNYHQLKLENLAVNVQEADAKLNPLNQTLPMV
LPVKVYPATAFGEVQYHKTPIRTVYIREEHTKALKMGNFKALVKDRRLNG
LFSFIKEENDTQKHPISQLRLRRELEIYQSLRVDAFKETLSLEEKLLNKH
TSLSSLENEFRALLEEWKKEYAASSMVTDEHIAFIASVANAFCANQYPFY
KEALHAPIPLFTVAQPTTEEKDGLGIAEALLKVLREYCEIVKSQIGSSLQ
KKLEELELGSQLHLPQVLADAVSRLVIGKFGDLTDNFSSPHARRIGLAGV
VMTTGTDVKDAKVICVSTGAKCINGEYLSDRGLALNDCHAEIVSRRSLLR
FLYTQLELYLNNEDDQKRSIFQKSERGGFRLKENIQFHLYISTSPCGDAR
IFSPHEAILEEPADRHPNRKARGQLRTKIEAGQGTIPVRNNASIQTWDGV
LQGERLLTMSCSDKIARWNVVGIQGSLLSIFVEPIYFSSIILGSLYHGDH
LSRAMYQRISNIEDLPPLYTLNKPLLTGISNAEARQPGKAPIFSVNWTVG
DSAIEVINATTGKGELGRASRLCKHALYCRWMRVHGKVPSHLLRSKITKP
NVYHETKLAAKEYQAAKARLFTAFIKAGLGAWVEKPTEQDQFSLT

An exemplary fusion protein used in the “CURE” system comprises or consists of SEQ ID NO: 22, SEQ ID NO: 23 or SEQ ID NO: 24. These fusion proteins are for instance expressible in mammalian producer cells using the “TC1332” plasmid from Tian Chi's laboratory (publicly available at Addgene, plasmid ref. #164874), the “TC1614” plasmid from Tian Chi's laboratory (publicly available at Addgene, plasmid ref. #164873), or the “TC1681” plasmid from Tian Chi's laboratory (publicly available at Addgene, plasmid ref. #164872).

SEQ ID NO: 22
MKRTADGSEFESPKKKRKVNIPALVENQKKYFGTYSVMAMLNAQTVLDHI
QKVADIEGEQNENNENLWFHPVMSHLYNAKNGYDKQPEKTMFIIERLQSY
FPFLKIMAENQREYSNGKYKQNRVEVNSNDIFEVLKRAFGVLKMYRDLTN
AYKTYEEKLNDGCEFLTSTEQPLSGMINNYYTVALRNMNERYGYKTEDLA
FIQDKRFKFVKDAYGKKKSQVNTGFFLSLQDYNGDTQKKLHLSGVGIALL
ICLFLDKQYINIFLSRLPIFSSYNAQSEERRIIIRSFGINSIKLPKDRIH
SEKSNKSVAMDMLNEVKRCPDELFTTLSAEKQSRFRIISDDHNEVLMKRS
SDRFVPLLLQYIDYGKLFDHIRFHVNMGKLRYLLKADKTCIDGQTRVRVI
EQPLNGFGRLEEAETMRKQENGTFGNSGIRIRDFENMKRDDANPANYPYI
VDTYTHYILENNKVEMFINDKEDSAPLLPVIEDDRYVVKTIPSCRMSTLE
IPAMAFHMFLFGSKKTEKLIVDVHNRYKRLFQAMQKEEVTAENIASFGIA
ESDLPQKILDLISGNAHGKDVDAFIRLTVDDMLTDTERRIKRFKDDRKSI
RSADNKMGKRGFKQISTGKLADFLAKDIVLFQPSVNDGENKITGLNYRIM
QSAIAVYDSGDDYEAKQQFKLMFEKARLIGKGTTEPHPFLYKVFARSIPA
NAVEFYERYLIERKFYLTGLSNEIKKGNRVDVPFIRRDQNKWKTPAMKTL
GRIYSEDLPVELPRQMFDNEIKSHLKSLPQMEGIDENNANVTYLIAEYMK
RVLDDDFQTFYQWNRNYRYMDMLKGEYDRKGSLQHCFTSVEEREGLWKER
ASRTERYRKQASNKIRSNRQMRNASSEEIETILDKRLSNSRNEYQKSEKV
IRRYRVQDALLFLLAKKTLTELADFDGEREKLKEIMPDAEKGILSEIMPM
SFTFEKGGKKYTITSEGMKLKNYGDFFVLASDKRIGNLLELVGSDIVSKE
DGSLQLPPLERLTLGSGSSMEASPASGPRHLMDPHIFTSNFNNGIGRHKT
YLCYEVERLDNGTSVKMDQHRGFLHNQAKNLLCGFYGRHAELRFLDLVPS
LQLDPAQIYRVTWFISWSPCFSWGCAGEVRAFLQENTHVRLRIFAARIYD
DDPLYKEALQMLRDAGAQVSIMTYDEFKHCWDTFVDHQGCPFQPWDGLDE
HSQALSGRLRAILQNQGN
SEQ ID NO: 23
MKRTADGSEFESPKKKRKVGSIEKKKSFAKGMGVKSTLVSGSKVYMTTFA
EGSDARLEKIVEGDSIRSVNEGEAFSAEMADKNAGYKIGNAKFSHPKGYA
VVANNPLYTGPVQQDMLGLKETLEKRYFGESADGNDNICIQVIHNILDIE
KILAEYITNAAYAVNNISGLDKDIIGFGKFSTVYTYDEFKDPEHHRAAFN
NNDKLINAIKAQYDEFDNFLDNPRLGYFGQAFFSKEGRNYIINYGNECYD
ILALLSGLAHWVVANNEEESRISRTWLYNLDKNLDNEYISTLNYLYDRIT
NELTNSFSKNSAANVNYIAETLGINPAEFAEQYFRFSIMKEQKNLGFNIT
KLREVMLDRKDMSEIRKNHKVFDSIRTKVYTMMDFVIYRYYIEEDAKVAA
ANKSLPDNEKSLSEKDIFVINLRGSFNDDQKDALYYDEANRIWRKLENIM
HNIKEFRGNKTREYKKKDAPRLPRILPAGRDVSAFSKLMYALTMFLDGKE
INDLLTTLINKFDNIQSFLKVMPLIGVNAKFVEEYAFFKDSAKIADELRL
IKSFARMGEPIADARRAMYIDAIRILGTNGTSKRTADGSEFESPKKKRKV
GPGSMEASPASGPRHLMDPHIFTSNFNNGIGRHKTYLCYEVERLDNGTSV
KMDQHRGFLHNQAKNLLCGFYGRHAELRFLDLVPSLQLDPAQIYRVTWFI
SWSPCFSWGCAGEVRAFLQENTHVRLRIFAARIYDDDPLYKEALQMLRDA
GAQVSIMTYDEFKHCWDTFVDHQGCPFQPWDGLDEHSQALSGRLRAILQN
QGN
SEQ ID NO: 24
MKRTADGSEFESPKAAAKVNIPALVENQKKYFGTYSVMAMLNAQTVLDHI
QKVADIEGEQNENNENLWFHPVMSHLYNAKNGYDKQPEKTMFIIERLQSY
FPFLKIMAENQREYSNGKYKQNRVEVNSNDIFEVLKRAFGVLKMYRDLTN
AYKTYEEKLNDGCEFLTSTEQPLSGMINNYYTVALRNMNERYGYKTEDLA
FIQDKRFKFVKDAYGKKKSQVNTGFFLSLQDYNGDTQKKLHLSGVGIALL
ICLFLDKQYINIFLSRLPIFSSYNAQSEERRIIIRSFGINSIKLPKDRIH
SEKSNKSVAMDMLNEVKRCPDELFTTLSAEKQSRFRIISDDHNEVLMKRS
SDRFVPLLLQYIDYGKLFDHIRFHVNMGKLRYLLKADKTCIDGQTRVRVI
EQPLNGFGRLEEAETMRKQENGTFGNSGIRIRDFENMKRDDANPANYPYI
VDTYTHYILENNKVEMFINDKEDSAPLLPVIEDDRYVVKTIPSCRMSTLE
IPAMAFHMFLFGSKKTEKLIVDVHNRYKRLFQAMQKEEVTAENIASFGIA
ESDLPQKILDLISGNAHGKDVDAFIRLTVDDMLTDTERRIKRFKDDRKSI
RSADNKMGKRGFKQISTGKLADFLAKDIVLFQPSVNDGENKITGLNYRIM
QSAIAVYDSGDDYEAKQQFKLMFEKARLIGKGTTEPHPFLYKVFARSIPA
NAVEFYERYLIERKFYLTGLSNEIKKGNRVDVPFIRRDQNKWKTPAMKTL
GRIYSEDLPVELPRQMFDNEIKSHLKSLPQMEGIDENNANVTYLIAEYMK
RVLDDDFQTFYQWNRNYRYMDMLKGEYDRKGSLQHCFTSVEEREGLWKER
ASRTERYRKQASNKIRSNRQMRNASSEEIETILDKRLSNSRNEYQKSEKV
IRRYRVQDALLFLLAKKTLTELADFDGERFKLKEIMPDAEKGILSEIMPM
SFTFEKGGKKYTITSEGMKLKNYGDFFVLASDKRIGNLLELVGSDIVSKE
DGSLQLPPLERLTLGSGSSMEASPASGPRHLMDPHIFTSNFNNGIGRHKT
YLCYEVERLDNGTSVKMDQHRGFLHNQAKNLLCGFYGRHAELRFLDLVPS
LQLDPAQIYRVTWFISWSPCFSWGCAGEVRAFLQENTHVRLRIFAARIYD
DDPLYKEALQMLRDAGAQVSIMTYDEFKHCWDTFVDHQGCPFQPWDGLDE
HSQALSGRLRAILQNQGN

Once the mammalian producer cells have produced the Retroviridae vectors, the latter may be harvested. This step is only optional, as the following steps may be performed directly in-bulk without the need to change the culture medium.

Means and methods for harvesting Retroviridae vectors are well known in the art. Typically, these include the retrieval of the mammalian producer cells' culture supernatant. Afterwards, one or several purification techniques can be carried out, such as, without limitation, a centrifugation, a clarification through a filter, a chromatography purification, a tangential flow filtration, and any combinations thereof.

Once the mammalian producer cells have produced the Retroviridae vectors, the latter are used to infect naïve mammalian reporter cells (i.e., cells which have never been in contact with the Retroviridae vectors).

The mammalian reporter cells may be human or non-human mammalian cells.

Examples of non-human mammalian cells have been detailed above in connection with mammalian producer cells, which also apply here mutatis mutandis.

In some preferred embodiment, the mammalian reporter cells are human cells.

The human reporter cells may be cells from an immortalized cell line, or they may be primary cells, e.g., derived from a tissue from a live or deceased human donor.

Examples of immortalized human cell lines have been detailed above in connection with mammalian producer cells, which also apply here mutatis mutandis.

In some preferred embodiments, the human reporter cells are selected from a group consisting of HEK293T cells, Jurkat cells and K-562 cells. In some preferred embodiments, the human reporter cells are HEK293T cells. In some preferred embodiments, the human reporter cells are Jurkat cells. In some preferred embodiments, the human reporter cells are K-562 cells.

In some embodiments, the mammalian reporter cells are infected with the Retroviridae vectors at a multiplicity of infection (MOI) ranging from about 0.1 to about 5, preferably at a MOI ranging from about 0.1 to about 2, such as, e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0, more preferably at a MOI of about 1±0.5.

The term “multiplicity of infection” or “MOI” refers to the ratio defined by the number of infectious Retroviridae vectors divided by the number of mammalian reporter cells in a given well or any other container.

In some embodiments, the mammalian reporter cells are infected with the Retroviridae vectors in the presence of hexadimethrine bromide. Hexadimethrine bromide acts by neutralizing the charge repulsion between Retroviridae vectors and sialic acid on the mammalian reporter cells' surface.

In some embodiments, the mammalian reporter cells are infected with purified Retroviridae vectors, i.e., the Retroviridae vectors produced by the mammalian producer cells were harvested and are used to infect naïve mammalian reporter cells in a fresh culture medium.

Alternatively, naïve mammalian reporter cells may be added to the mammalian producer cells' medium (optionally replenished) after the Retroviridae vectors have been produced and released in the medium, for in-bulk process. This can be in particular desirable when the method of the invention is automated.

Once the mammalian reporter cells were infected with the Retroviridae vectors, they are incubated under conditions allowing for SoI expression.

In some embodiments, the mammalian reporter cells are cultured in a culture medium suitable for their growth. It shall be understood that the skilled artisan readily knows which culture medium is suitable depending on the cell type. Non-limiting examples of culture medium include Minimum Essential Medium (MEM), Eagle's minimal essential medium (EMEM), Dulbecco's Modified Eagle's Medium (DMEM), RPMI 1640 medium, Iscove's Modified Dulbecco's Medium (IMDM), Roswell Park Memorial Institute medium (RPMI), Ham's tissue culture medium, William's Medium, and F-12 medium.

In some embodiments, the culture medium is supplemented with nutrients. Non-limiting examples of nutrients include sugars, amino acids, proteins, vitamins, fatty acids, lipids, and combinations thereof. An example of suitable sugar is glucose. An example of suitable amino acid is glutamate or a derivative thereof, e.g., glutamine.

In some embodiments, the culture medium is further supplemented with growth factors. The skilled artisan knows how to select suitable growth factors depending on the cell type. In some embodiments, the growth factors are provided in a serum. Non-limiting examples of serum include bovine serum, fetal bovine serum (FBS), goat serum, horse serum, sheep serum, rabbit serum and chicken serum.

In some embodiments, the culture medium is further supplemented with agents inhibiting the growth of, and/or eliminating, microorganisms (e.g., bacteria, fungi, mycoplasmas and the like), typically an antibiotic agent or an antifungal agent, or combinations thereof. Example of suitable antibiotic agents include penicillin and streptomycin.

In some embodiments, the culture medium comprises a pH buffer, preferably a pH buffer suitable for atmospheric condition comprising 5% CO₂.

In some embodiments, the culture medium is suitable for culturing HEK293 cells, preferably HEK293T cells. In some embodiments, the culture medium is Dulbecco's modified Eagle medium. In some embodiments, the culture medium is Dulbecco's modified Eagle medium supplemented with high glucose, 10% fetal bovine serum, 2 mM glutamine, 100 U penicillin and 0.1 mg/mL streptomycin, as exemplified in Example 1 below.

In some embodiments, the culture medium is suitable for culturing Jurkat cells and/or K-562 cells. In some embodiments, the culture medium is RPMI 1640 medium. In some embodiments, the culture medium is RPMI 1640 medium supplemented with 10% FBS, 1% penicillin-streptomycin and 1% GlutaMAX, as exemplified in Example 1 below.

In some embodiments, the mammalian reporter cells are incubated at a temperature ranging from about 30° C. to about 44° C., preferably from about 35° C. to about 39° C., more preferably from about 36° C. to about 38° C., even more preferably at about 37° C.

In some embodiments, the mammalian reporter cells are incubated under a proportion of CO₂ in air ranging from about 1% to about 9%, preferably from about 2% to about 8%, more preferably from about 3% to about 7%, even more preferably from about 4% to about 6%, even more preferably at about 5%.

Depending on the type of SoI the biological activity of the product it encodes can be measured by various means and methods, either directly or indirectly, through a phenotype of the reporter mammalian cells caused by the expression of the SoI mutant.

By measuring the biological activity of an SoI product, it is possible to select those SoI mutants which provide a desirable function, for instance, an improved biological activity compared to the wild-type SoI product. The skilled artisan will understand that an improvement of biological activity is not necessarily the desired endpoint, which could alternatively be a reduced if not abolished biological activity, or a different biological activity than observed with the wild-type SoI product.

In some embodiments, the phenotype caused by the expression of the SoI mutant, either directly or indirectly, may include, without limitation, a transcriptional activity, a translational activity, an enzymatic activity, a binding activity, a drug resistance, a fluorescence, etc.

It will be understood that an aspect of the method of the invention aims to generate mutants that have altered functional properties compared to the unaltered sequence (or wild-type, WT). The method thus enables synthetic evolution of virtually any functionally active nucleic acid sequence on a functional rather than a structural basis.

Some examples of means and methods to measure the biological activity of an SoI product are given in the Example section. By ways of example, when the SoI encodes an enzyme, its biological activity can be measured directly by a corresponding enzymatic assay. When the SoI encodes a regulatory element (for instance, a promoter), its biological activity can be measured indirectly though the expression of a gene or activity of a gene's product, when said gene is under the control of this regulatory element. Thus, illustratively, the method of the invention can be applied to a SoI that is and/or encodes a promoter in order to modulate its function by either increasing or decreasing the expression of a gene under the control of this promoter.

The mammalian reporter cells may express an exogenous protein or fragment thereof which acts as a reporter for the selection of SoI mutants with a desired biological activity. The skilled artisan will readily understand that the reporter should be selected based on the nature of the SoI product, so that the phenotype caused by the reporter correlates directly with the biological activity of the SoI product.

This reporter may be, e.g., an enzyme, a fluorescent protein, a bioluminescent protein, a chromogenic protein, a surface marker, a viability marker, etc.

Some examples of fluorescent proteins include, without limitation, green fluorescent protein (GFP), enhanced GFP (eGFP), red fluorescent protein (RFP), yellow fluorescent protein (YFP), DsRed, mCherry, mOrange, and mRuby.

Means to detect fluorescence are known in the art and comprise, without limitation, fluorescence microscopy (e.g., epifluorescence microscopy, confocal microscopy, light sheet microscopy), plate reader, fluorometer (or fluorescence spectrophotometer), cell sorting and in particular fluorescence-activated cell sorting (FACS).

In some embodiments, the reporter is an enzyme, and the product resulting from the reaction catalyzed by the enzyme is detectable. The reaction catalyzed by the enzyme may produce light (e.g., luciferase or horseradish peroxidase), or a colored compound (e.g., β-galactosidase), or may induce post-translational modifications of proteins (e.g., biotinylation) that are later detected by suitable means (e.g., detection of biotin by an anti-biotin antibody, or by affinity with streptavidin, combined with any suitable analytical technique).

In some embodiments, the reporter is a bioluminescent protein (e.g., aequorin).

In some embodiments, the reporter is a chromogenic protein (e.g., Rtm5 protein or ultramarine protein).

In some embodiments, the reporter is surface marker (e.g., a receptor), and the surface marker is recognized by, e.g., an antibody or a ligand modified to be detectable (fluorescence, radioactive isotope, fusion with an enzyme and the like).

In some embodiments, the reporter is a viability marker, for instance a viability marker comprising a tag (e.g., a fluorescent tag, or a tag for affinity detection or purification).

In some embodiments, the reporter may be detected regardless of its intrinsic properties by a selective protein detection method, such as, e.g., Western-Blot or liquid chromatography-mass spectrometry (LC-MS) or variants thereof.

In some embodiments, the SoI encodes a fragment of a protein (e.g., a fragment of a fluorescent protein), and the mammalian reporter cells expresses another fragment of said protein. The fragments expressed by both the SoI and the mammalian reporter cells may then be assembled to form a full functional and detectable protein.

Once SoI mutants which provide a desirable function have been selected, it is possible to reiterate the full method described herein, to further evolve the SoI mutants.

In this case, the sequence of the selected SoI mutant serves as new SoI in the second nucleic acid molecule encoding the recombinant expression cassette, and the method is reiterated with the co-transfection of mammalian producer cells, incubation, optionally harvesting of Retroviridae vectors, infection of mammalian reporter cells, incubation and selection of one or several SoI mutants.

The number of iterations is not limited by any factor, and can range from 1 to several tens.

In some embodiments, the method of the invention does not rely solely on the basal mutagenesis rate of Retroviridae vectors for inducing mutations in the SoI. In some embodiments, the method of the invention comprises at least one mean for inducing mutations in the SoI other than the basal mutagenesis rate of Retroviridae vectors.

In certain embodiments, the method of the invention is not a method of drug resistance screening. In certain embodiments, the method of the invention is not a method of screening for alleles of genes associated with resistance to drugs. In certain embodiments, the method of the invention is not a method of exploring gene function.

The present invention further relates to a mutant of an SoI with a desired biological activity, obtained or obtainable by the method of the invention described herein. It also relates to any nucleic acid molecule or protein encoded by such mutant of the SoI.

The present invention further relates to a mammalian producer cell stably expressing a deaminase, as described herein. It also relates to a mammalian producer cell stably expressing a fusion protein comprising (i) a deaminase, a catalytically active domain thereof, or a mutant thereof which retains its deaminase activity or has an improved deaminase activity as compared to the corresponding wild-type deaminase as described herein, and (ii) a RNA targeting CRISPR effector as described herein.

### BRIEF DESCRIPTION OF THE DRAWINGS

FIG. 1 is a scheme illustrating the retroviral synthetic evolution (RSE) platform described herein.

FIGS. 2A-D are a combination of schemes, histograms and graphs showing the evolution and selection of mutant GFP genes via the retroviral synthetic evolution (RSE) platform described herein. FIG. 2A illustrates the schematics of the process. Initially, Retroviridae vectors are produced by co-transfecting producer cells with a packaging plasmid, an envelope plasmid and a transfer plasmid, the latter comprising the GFP gene to be evolved. The supernatant, comprising the Retroviridae vectors with the evolved GFP gene, is harvested 48 h later and used to infect naïve reporter cells. These cells express the evolved GFP gene and can be sorted, e.g., by FACS, for GFP expression. The best evolved GFP gene is retained and used for a new iteration of the method. FIG. 2B shows the increase in fluorescence signal detected after 5 iterations of the method. FIG. 2C shows the mutation frequencies across the promoter region of the GFP gene after 5 iterations of the method. FIG. 2D shows the titter comparison of wild-type Retroviridae vector [WT] and three mutants selected after 5 iterations of the method ([1], [2] and [3]).

FIG. 3 is a histogram showing the tuning mutagenesis across lentiviral payload.

FIGS. 4A-B are a scheme and a histogram illustrating focused diversification. The scheme of FIG. 4A shows a way of achieving focused diversification, i.e., using a Cas13-guided deaminase targeted to a specific region of the sequence of be evolved by means of a guide RNA. The length of the guide RNA can be variable, determining the targetable window. This enables directing mutagenesis to specific regions of the sequence of be evolved. FIG. 4B shows mutation frequencies in the DNA binding domain of a synthetic nuclease. The 90-bp grey bar shows the targeted region.

FIGS. 5A-C are schemes and histograms illustrating the evolution of the gag-pol genes using the RSE platform. FIG. 5A is a schematic representation of the cycling process. The gag-pol genes are encoded in the transfer plasmid together with an RFP gene. The supernatant, comprising the Retroviridae vectors with the evolved gag-pol genes, is harvested 48 h later and used to infect naïve reporter cells. Functional gag-pol genes allow reporter cells to express RFP, which is used as reporter to sort the best evolved gag-pol genes, e.g., by FACS. FIG. 5B shows the viral titter across cycles measured as percentage of RFP cells after transduction of harvested supernatant from the previous cycle. FIG. 5C shows the percentage of RFP of isolated variants from cycle 6 compared to the original vector [WT].

FIGS. 6A-C is a combination of schemes and histogram illustrating the evolution of a programmable transposase. FIG. 6A shows a schematic overview of the cycling. The transposase gene is encoded in the transfer plasmid and diversified using focused mutagenesis with Cas13-deaminase targeting. The supernatant, comprising the Retroviridae vectors with the evolved transposase gene, is harvested 48 h later and used to infect naïve reporter cells that contain an integrated reporter with a half-GFP gene. Functional transposase genes allow the transposition of a transposon DNA comprising the other half of the GFP gene, allowing the cells to express GFP, which is then used as reporter to sort the best evolved transposase genes, e.g., by FACS. FIG. 6B shows an alternative integrated reporter comprising a half viral envelope gene. The envelope gene is reconstituted upon infection of the reporter cells with the Retroviridae vectors, allowing viral production by the reporter cells themselves. This alternative by-passes the need of cell sorting and increases screening power. FIG. 6C shows the reconstitution of the viral envelope glycoprotein VSV-G upon targeted integration followed by viral production with the infected cell population using the split reporter of FIG. 6B. This strategy ensures that only evolved transposases that are functional are propagated, by-passing the need of sorting and increasing screening power.

FIGS. 7A-B is a combination of schemes and histogram illustrating the evolution of a nuclease (Cas9). FIG. 7A shows a schematic overview of the cycling process. The nuclease gene is encoded in the transfer plasmid. The supernatant, comprising the Retroviridae vectors with the evolved nuclease gene, is harvested 48 h later and used to infect naïve reporter cells that contain an integrated reporter with an out-of-frame viral envelope gene [Env*] followed by a fluorescent marker [RFP*] with an upstream nuclease target site [t]. Functional nuclease genes allow the cleavage of the integrated reporter at the target site, inducing indel formation by non-homologous end joining, and recovery of the frame of the viral envelope gene and fluorescent marker. FIG. 7B shows the frame recovery with two evolved nucleases, leading to the expression of a functional viral envelope which allows propagation of the functional mutants to the next cycles, and expression of a functional fluorescent marker.

FIG. 8 shows deaminase mutagenesis. RSE vector encoding a Blue fluorescent protein (BFP) and mutagenesis components are packaged using RSE system. BFP to GFP conversion models the attempt to find an improved variant, being blue fluorescence the starting state and green fluorescence the improved state. M230I RT mutation system and deaminase guided focused mutagenesis methods are compared.

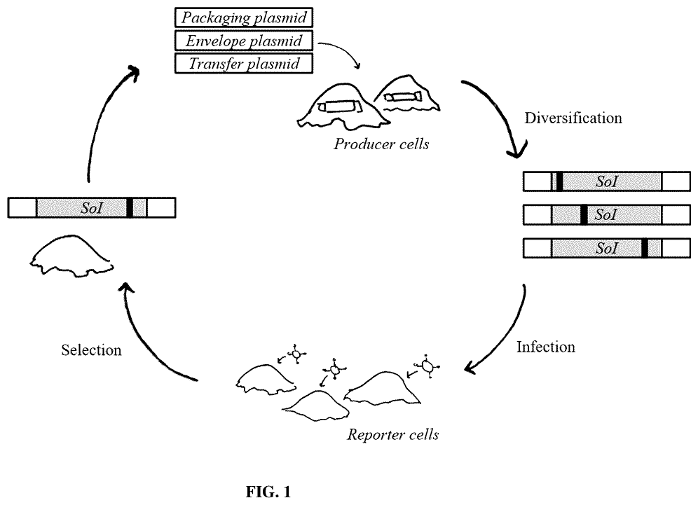

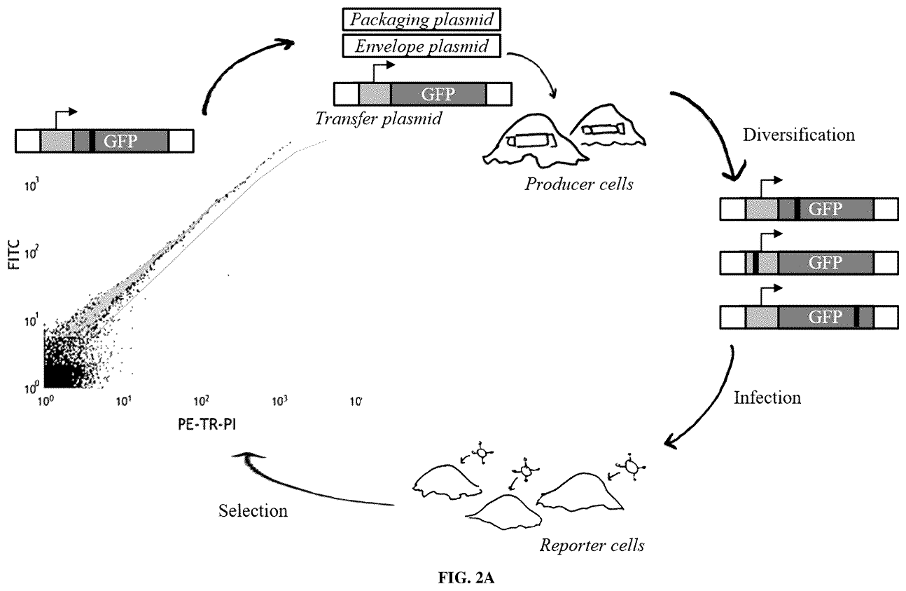

### EXAMPLES

The present invention is further illustrated by the following examples.

### Example 1

### Materials and Methods

### Plasmid Constructions

The following plasmids were obtained from Addgene:
psPAX2 (plasmid #12260),
pCMV-VSV-G (plasmid #8454),
pMDLg/pRRE (plasmid #12251),
pRSV-Rev (plasmid #12253), and
pSICO (plasmid #11578).

Mutations in the reverse transcriptase sequence of the psPAX2 packaging plasmid were obtained by performing site-directed mutagenesis with QuikChange Lightning Multi SDM Kit (Agilent #210513). All the remaining vectors were built following golden gate assembly using Esp3I restriction enzyme and T4 ligase.

### Cell Culture

HEK293T cell line (ATCC CRL-3216) was grown in Dulbecco's modified Eagle medium (DMEM), supplemented with high glucose (Gibco, Thermo Fisher), 10% fetal bovine serum, 2 mM glutamine and 100 U penicillin/0.1 mg/mL streptomycin.

K-562 cell lines (ATCC CRL-3343) and Jurkat-T cell lines (clone E6-1 ATCC IB-152) were grown in RPMI 1640 medium (Gibco), supplemented with 10% FBS, 1% penicillin-streptomycin (Gibco) and 1% GlutaMAX (100×) (Gibco).

### Lentiviral Production and Tittering

Lentiviral vectors (LV) were produced following protocol available at www.addgene.org/protocols/Lentivirus-production, with the following modifications.

Cells were produced in 10-cm dishes seeded with 4.9×10⁶ HEK293T cells a day prior transfection using 0.72 pmol of pCMV-VSV-G envelope plasmid, 1.64 pmol of pSICO or pRRL vector payloads (transfer plasmid) and 1.30 pmol psPAX2 plasmid or psPAX2 mutants. Plasmids were mixed in 500 μL Optimem and 100 mg polyethyleneimine (PEI).

Two days after plasmid transfection, supernatant was harvested and filtered using through a 0.45-μM filter. In some cases, viral vectors were centrifuged overnight at 4000×g at 4° C. Supernatant was discarded and LV particles were resuspended to achieve 100× vector concentration.

### Reporter Cell Line Generation

Reporter sequences were stably integrated in HEK293T cells by transfecting sleeping beauty or piggyBac transposase plasmids and corresponding transposon cargo genes encoding the reporter construct.

Three days after transfection, cells were selected with antibiotics for 2 weeks. For monoclonal cell line generation, cells were sorted in single wells 7 days after transfection and expanded with antibiotic selection.

### Assessment of Mutagenesis Levels

For assessing diversification increase, lentiviral vectors were produced as described above, replacing psPAX2 with respective psPAX2 containing reverse transcriptase mutant.

10 μL of vector were used for infecting HEK293T cells at an approximate MOI of 1. Genomic DNA of infected cells was extracted 48 hours post-infection using the DNeasy Blood & Tissue Kit (Qiagen's ref. 69504) and the sequence of interest to be evolved was amplified and sequenced with an Illumina Miseq 2×250PE sequencer.

For assessing drug-induced mutagenesis, 5-azacytidine (Sigma's ref. A2385-100MG) or 5-hydroxydeoxycytidine (5-OH-dC) was added to the cells at different doses and sequenced as described below.

For assessing focused editing, cells were transfected with Cas13-deaminase and gRNAs of different lengths designed to target different regions of the sequence of interest to be evolved.

### Sequencing & Validation of Evolved Phenotypes:

Genomic DNA from infected cells was extracted after desired number of cycles. The sequence of interest was amplified with primers containing Esp3T overhangs and cloned in a plasmid. Single colonies were sequenced and individually transfected to evaluate activity in their specific reporter cells. Amplified sequences of interest were also sequenced using Pacbio SMRT I flow cell.

### Results

### GFP Gene Optimization Via RSE Platform

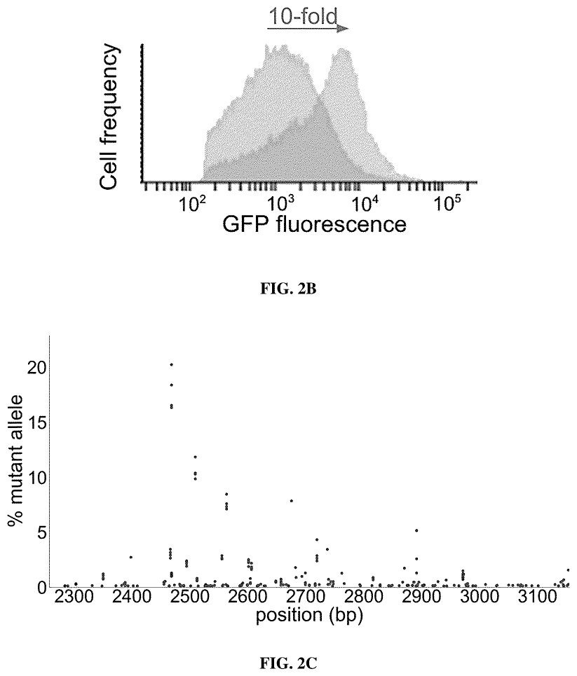

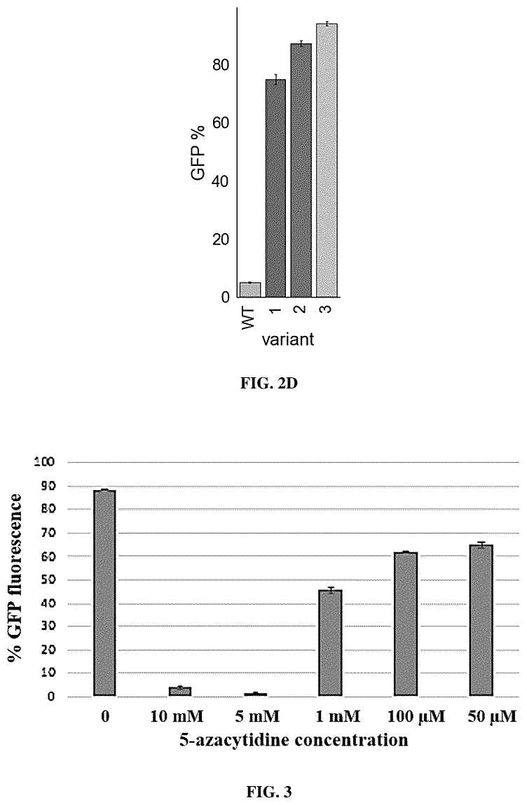

To demonstrate implementation of synthetic evolution in mammalian cells using the retroviral system of the invention, a GFP expressing vector was cycled for 5 consecutive generations, optimizing its expression and packaging in HEK293T cells. Viral particles were generated by expressing packaging components (lentiviral gagpol and vsv-g envelope) together with a transfer plasmid containing an LTR-flanked GFP gene (FIG. 2A). Infected GFP-expressing cells were selected by sorting, in order to enrich improved genotypes.

HIV reverse transcriptase error rate is 1.4×10⁻⁵, suggesting that an evolution campaign could be performed without necessitating any additional mutagenesis. The transfer plasmid contains a chimeric CMV-LTR sequence that drives expression and acts as an operational vector sequence. The synthetic origin of the CMV-LTR suggested that this vector could further adapt to the specific environment in which viral particles are produced.

Expression of GFP upon infection with the evolving population of LVs increased after 5 cycles (FIG. 2B), suggesting that the population was evolving towards the driven phenotype.

RNA sequencing of bulk particles from cycle 5 showed substantial mutagenesis across CMV-LTR promoter sequence compared to cycle 1 (FIG. 2C). Isolation and testing of evolved mutants revealed an increase in expression and titter (FIG. 2D). Sanger sequencing of top performers revealed an intron in the CMV sequence.

### Diversification Strategies

Several ways were explored to control the mutagenesis rate.

A first tested aspect was the addition, in the producer cells, of the nucleoside analogs 5-azacytidine (5-aza-C) and 5-hydroxydeoxycytidine (5-OH-dC), which were described in the art to increase the viral reverse transcriptase error rate. As seen on FIG. 3, 5-aza-C had an effect on the viral titter. However, 5-10 mM 5-aza-C seemed too highly concentrated and resulted mainly in a near-complete loss of functionality of the GFP gene.

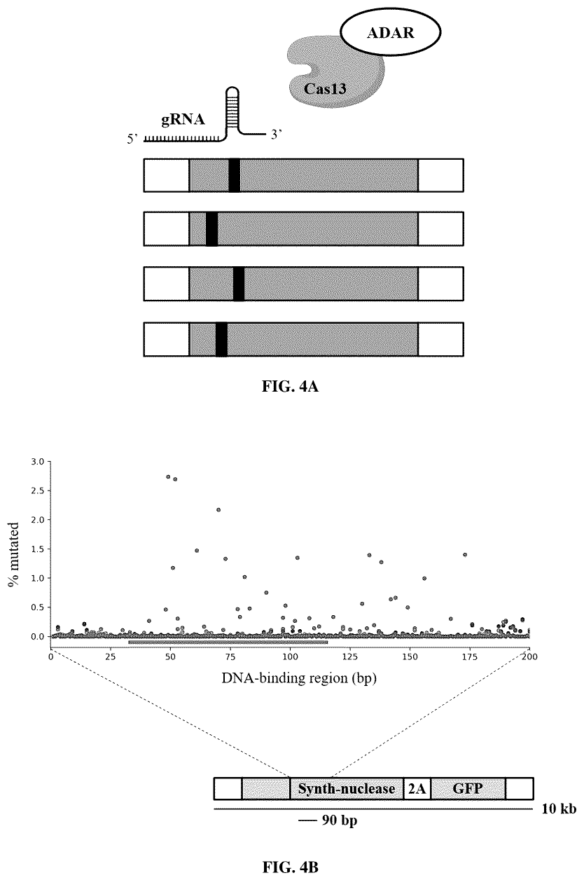

A second tested aspect was “focused diversification”, which enables user-guided mutagenesis across a region of a sequence of interest, allowing the creation of tailored molecular populations and maximizing the variants that can be functional if prior knowledge is used to guide diversification. We tested whether the RNA editing system RESCUE (Abudayyeh et al., 2019. Science. 365(6451):382-386) could be adapted for focused diversification with our RSE platform. dRanCas13b gRNAs targeting the DNA-binding domain of a piggyBac protein encoded in the Retroviridae vectors was designed (FIG. 4A). C→T and A→G substitutions were observed at the target sequence and also downstream of the target site, probably corresponding to the steric properties of the dRanCas13b-ADAR2 fusion protein (FIG. 4B).

### RSE-Improved Gag-Pol Packaging System

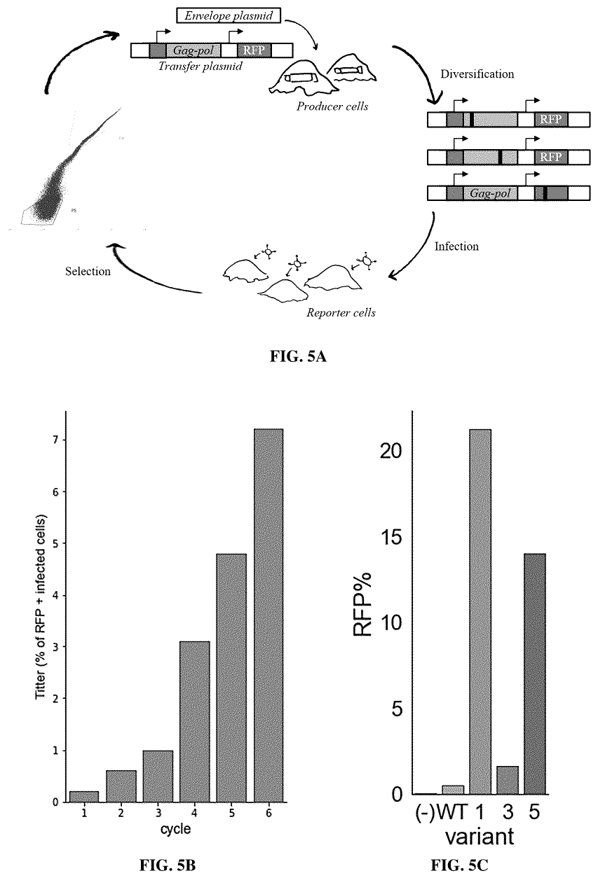

Given the observed titter increase after vector evolution, it was attempted to optimize the packaging components by adding the gag-pol genes in the sequence to be evolved in the transfer plasmid, and implementing an evolution campaign (FIG. 5A).

An increase in vector titter was observed with each evolution cycle (FIG. 5B).

Isolation of the evolved mutants revealed a titter increase, and the sequencing of the top mutants revealed mutations across the vector sequence (FIG. 5C).

### Evolution of a Programmable Transposase

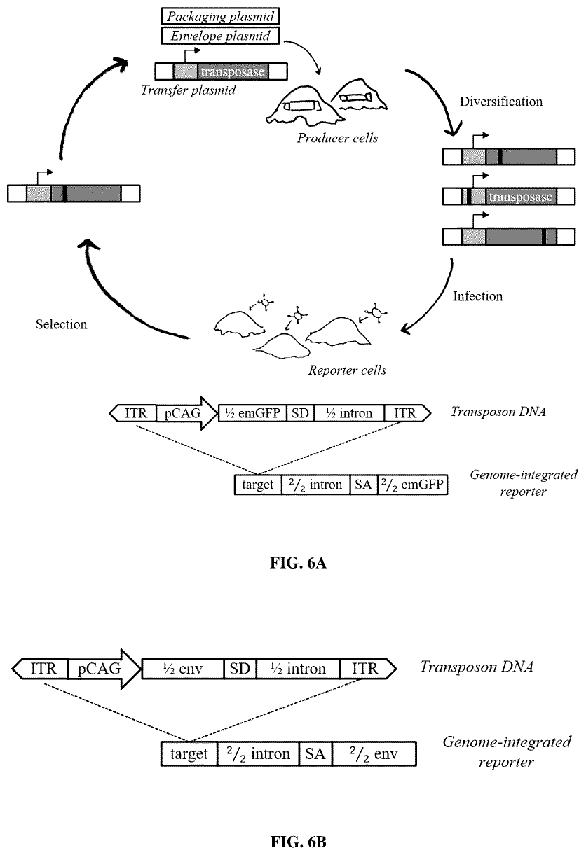

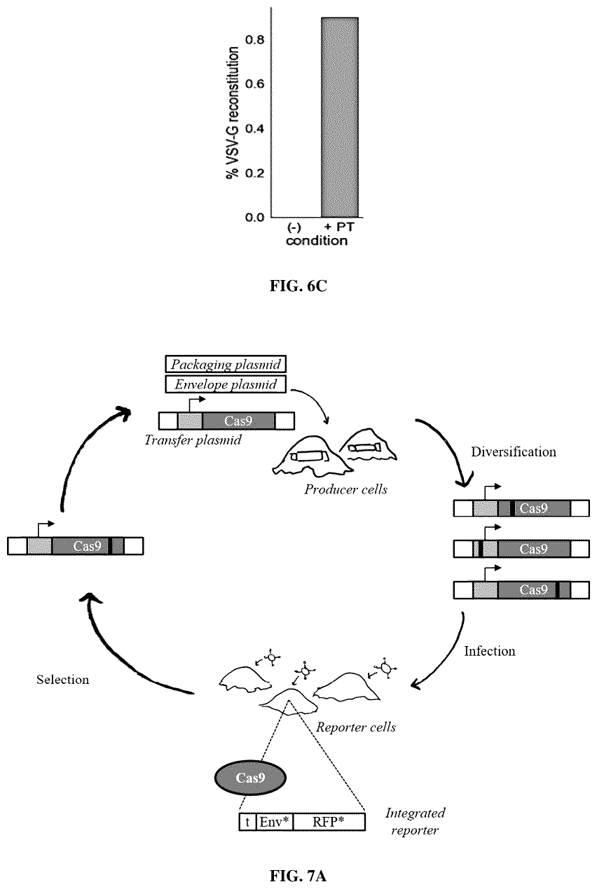

We aimed at evolving a programmable integrase/transposase using the RSE platform, as shown in FIG. 6A. Functional mutants of the programmable integrase/transposase could be detected, by reconstitution of a detectable marker such as GFP using reporter mammalian cells comprising a genome-integrated reporter (with a split GFP gene) in the presence of a transposon DNA (comprising the other half of the split GFP gene). Alternatively, reconstitution of an antibiotic resistance was also envisaged. Selection of functional mutants was achieved by retaining those reporter cells expressing the marker. Selected reporter cells were then used for viral production, and further rounds of random and/or focused mutagenesis using the RSE platform.

As an alternative to fluorescence or antibiotic selection, we also developed reporter mammalian cells comprising a split viral envelop gene, which could be reconstituted in the presence of a transposon DNA comprising the other half of the envelop gene (FIG. 6B). In this situation, only cells with functional mutants experienced Env reconstitution and thus became competent for viral production, passing the functional mutants to the next generation. As shown in FIG. 6C, a mutant with increased activity was obtained, by coupling targeted integration to viral production.

### Evolution of an Ancestral Nuclease

Viral fitness can be coupled to a desired enzyme function in several distinct ways, e.g., DNA sequence modification by frame restoration, stop codon removal, increase in expression by modifying cis-acting or trans-acting DNA regulatory sequence, etc.

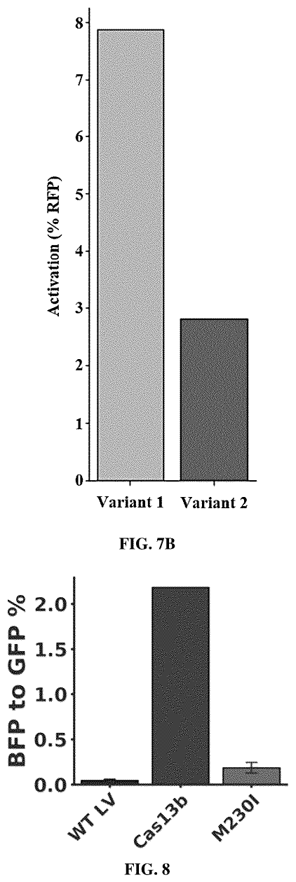

As an illustrative example, frame restoration of a viral component could be achieved, such as, of the viral envelope gene VSV-G (or “Env”) (FIG. 7A), capsid protein, accessory proteins or any other viral packaging proteins.

In this example, out-of-frame VSV-G was stably introduced in a mammalian HEK293T reporter cell line, with a target site upstream. Editing by a nuclease at the target site provided VSV-G frame restoration that could be detected by Western blotting. A fluorescent reporter was also reconstituted together with VSV-G for cytometry measurement. As seen on FIG. 7B, exemplary mutants of a nuclease could be obtained and their increase in functionality detected by frame restoration and fluorescence measurement.

### Discussion

Currently, no evolution platform exists in mammalian cells on an industrial scale. The present invention discloses a unique method of directed evolution for designing and screening up to 10 million mutants of sequences of interest in mammalian systems.

Current solutions are performed by single-well manual or automated screens (10² to 10³ variants can be tested), and are based in non-focused diversification strategies, which limits discovery.

### Example 2

### Materials and Methods

Cells were transfected as explained above (see Example 1). The vector used is a lentiviral vector for BFP (blue fluorescent protein) of SEQ ID NO: 25.

Cells produce viral particles encoding the gene of interest (GOI) upon transfection of RSE vector producing components, consisting of packaging, envelope, transgene and mutagenesis plasmids (see Example 1).

A focused mutagenesis strategy directed at a region of potential improvement of the sequence (i.e., catalytic site) is employed by transfecting RNA/DNA guided deaminase targeting the GOI sequence

BFP to GFP conversion is used to model chances of obtaining a desired population, obtained by “successful” mutations in the ROI

Activity of the GOI is measured by coupling the activity of the GOI fluorescence, resistance, or viral replication.

### Results

FIG. 8 shows increased fluorescence activity of the pool of sequences obtained with deaminase guided by Cas13b, compared to the fluorescence activity of the pool of sequences obtained with the error-prone error prone reverse transcriptase M230I. Focused mutagenesis increases the proportion of sequences with the desired phenotype (GFP), thus increasing the chances of discovering an improved phenotype compared to M230I mutagenesis system.

1-15. (canceled)

16. A method of synthetic evolution of a nucleic acid sequence of interest (SoI), comprising:
(a) co-transfecting mammalian producer cells with
(i) a first nucleic acid sequence encoding a Retroviridae genome comprising at least the gag and pol genes,
(ii) a second nucleic acid sequence encoding a recombinant expression cassette comprising, from 5′ to 3′:
a 5′ long terminal repeat (5′ LTR),
a packaging sequence,
the SoI to be evolved,
a 3′ long terminal repeat (3′ LTR);
(b) incubating the mammalian producer cells under conditions allowing for mutation of the SoI and production of Retroviridae vectors;
(c) harvesting the population of Retroviridae vectors produced after step (b);
(d) infecting mammalian reporter cells with the population of Retroviridae vectors of (c);
(e) incubating the mammalian reporter cells under conditions allowing for SoI expression; and
(f) selecting one or several mutants of the SoI with a desired biological activity;
wherein steps (a)-(f) are reiterated until a mutant of the SoI with the desired biological activity is obtained.

17. The method according to claim 16, wherein at step (a) said mammalian producer cells are further co-transfected with (iii) a third nucleic acid sequence encoding a viral envelope glycoprotein.

18. The method according to claim 16, wherein mammalian producer cells are incubated at step (b) in the presence of a mutagen selected from the group comprising irradiation, DNA intercalating agents, reactive oxygen species, nucleic acids, and mutagenic drugs.

19. The method according to claim 18, wherein the mutagen is a nucleoside analog.

20. The method according to claim 18, wherein the mutagen is 5-hydroxy-2′-deoxycytidine (5-OH-dC) or 5-azacytidine (5-aza-C).

21. The method according to claim 16, wherein mammalian producer cells are incubated at step (b) in the presence of a single-stranded or double-stranded RNA-specific deaminase or a nucleic acid coding therefor.

22. The method according to claim 16, wherein mammalian producer cells are incubated at step (b) in the presence of an RNA-specific adenosine deaminase or an RNA-specific cytidine deaminase.

23. The method according to claim 21, wherein the deaminase is fused to a programmable RNA-guided protein and the mammalian producer cells are further incubated at step (b) with a guide RNA comprising a first region at least partially complementary to a region of interest of the SoI and a second region capable of interacting with the programmable RNA-guided protein.

24. The method according to claim 16, wherein the pol gene expresses an error-prone reverse transcriptase/RNaseH mutant.

25. The method according to claim 16, wherein the pol gene expresses an error-prone reverse transcriptase/RNaseH mutant comprising at least one mutation selected from the group consisting of Y115A, M184V, M184I, Q151M, M230I and Y501W, said position number corresponding to the amino acid number of SEQ ID NO: 1.

26. The method according to claim 16, wherein the Retroviridae vector is a lentiviral vector.

27. The method according to claim 16, wherein the Retroviridae vector is a lentiviral vector selected from the group consisting of human immunodeficiency virus 1 (HIV-1), human immunodeficiency virus 1 (HIV-2), simian immunodeficiency virus (SIV), feline immunodeficiency virus (FIV), bovine immunodeficiency virus (BIV), puma Lentivirus (PLV), equine infectious anemia virus (EIAV), caprine arthritis encephalitis virus (CAEV), Visna-maedi virus, and Jembrana disease virus.

28. The method according to claim 16, wherein the Retroviridae vectors are not self-inactivating vectors.

29. The method according to claim 16, wherein the mammalian producer cells and/or the mammalian reporter cells are human cells.

30. The method according to claim 16, wherein the SoI is a coding nucleic acid sequence or a non-coding nucleic acid sequence.

31. The method according to claim 30, wherein the SoI is a regulatory element or expresses a non-coding RNA.

32. The method according to claim 30, wherein the SoI is a coding nucleic acid sequence encoding a peptide or protein.

33. The method according to claim 16, wherein selection of mutants of the SoI at step (f) is performed based on a measurable phenotype directly or indirectly caused by the SoI mutant in the mammalian reporter cells.

34. The method according to claim 16, wherein the mammalian reporter cells at steps (d) and (e) express an exogenous protein or fragment thereof acting as reporter for the selection of mutants of the SoI with a desired biological activity.

35. A mutant nucleic acid sequence of interest obtainable by the method according to claim 16.


SEQUENCE LISTING (25 sequences)

SEQ ID NO: 1 - protein, 566 aa, Human immunodeficiency virus 1
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGID

SEQ ID NO: 2 - protein, 566 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAAFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGID

SEQ ID NO: 3 - protein, 566 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYVDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGID

SEQ ID NO: 4 - protein, 566 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYIDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGID

SEQ ID NO: 5 - protein, 566 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPMGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGID

SEQ ID NO: 6 - protein, 566 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWIGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGID

SEQ ID NO: 7 - protein, 566 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQWALGIIQAQPDQSESELVNQIIEQLIKKEKVYLAWVPAHKGIGGNEQVDKLVSAGIRKVLFLDGID

SEQ ID NO: 8 - protein, 741 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 9 - protein, 750 aa, Homo sapiens
MASSTTGPLHMGTFFSIMGRRYKRRRKKRSERKDRNSLRQSRNPQKYFAMDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 10 - protein, 946 aa, synthetic construct
MNNKRKTKAKAAGLKSVFFDQKQAVLTTFAKGNNSQIEKKVVNSEVKDLRQPPAFDLELKEKTFYISGKNNINTSRENPLASASLPLSKRQRIRAERIKRAREENRPYHNVKRVGEDDLRAKADLEKHYFGKEYSDNLKIQIIYNILDINKIISPYINDIVYSMNNLARNDEYIDGKIDVIGSLSSTTDYSSFMSPNKDLEKEKKFSFHRENYKKFVEASKPYMRYYGKVFIRDVKKSKLSTGKGEKIEVMYRSDEEIFTIFQILSYVAQSIMANDIGNKSSILAIEKYPARFVGFLSDLLKTKTNDVNRMFIDNNSQTNFWVLFSIFGLQDHTSGADKICRNFYDFVIKADSKNLGFSLKKIRELMLDLPNANMLRDHQFDTVRSKFYTLLDFIIYQHYLEEKSRIDNMVEKLRMTLKEEEKEVLYAAEAKIVWNAIGAKVINKLVPMMNGDALKEIKRKNRDRKLPQSVIATVQVNSDANVFSGLIYFLTLFLDGKEINEMVSNLITKFENIDSLLHVDREIYKSDEKDLDLEIEKLALFFKGVVRPNAKTDTGAGEISKSFSIFQSAERIIEELKFIKNVTRMDNEIFPSEGVFLDAANVLGVRGDDFDFSNEFVGDDLHSDANKKIINKINGTKEDRNLRNFIINNVVKSRRFQYIARHMNTHYVKQLANNETLNRFVLNKMGDAKIINRYYESISGNTPNIEVRSQIDYLVKRLRSFSFEDLNDVKQKVRPGTNESIEKEKKKALVGLCLTIQYLVYKNLVNINARYTTAFYCLERDSKLKGFGVDVWRDFESYTALTNHFIKEGYLPVRKAEILRANLKHLDCEDGFKYYANQVTALNAIRVAYKYINEIKSVHSYFALYHYIMQRHLYDSLQAKAKDSSGFVIDALKKSFEHKIYSKDLLHVLHSPFGYNTARYKNLSIEALFDKNESRPEVNPLSTND

SEQ ID NO: 11 - protein, 954 aa, synthetic construct
MGKKIHARDLREQRKTDRTEKFADQNKKREAERAVPKKDAAVSVKSVSSVSSKKDNVTKSMAKAAGVKSVFAVGNTVYMTSFGRGNDAVLEQKIVDTSHEPLNIDDPAYQLNVVTMNGYSVTGHRGETVSAVTDNPLRRFNGRKKDEPEQSVPTDMLCLKPTLEKKFFGKEFDDNIHIQLIYNILDIEKILAVYSTNAIYALNNMSADENIENSDFFMKRTTDETFDDFEKKKESTNSREKADFDAFEKFIGNYRLAYFADAFYVNKKNPKGKAKNVLREDKELYSVLTLIGKLAHWCVASEEGRAEFWLYKLDELKDDFKNVLDVVYNRPVEEINNRFIENNKVNIQILGSVYKNTDIAELVRSYYEFLITKKYKNMGFSIKKLRESMLEGKGYADKEYDSVRNKLYQMTDFILYTGYINEDSDRADDLVNTLRSSLKEDDKTTVYCKEADYLWKKYRESIREVADALDGDNIKKLSKSNIEIQEDKLRKCFISYADSVSEFTKLIYLLTRFLSGKEINDLVTTLINKFDNIRSFLEIMDELGLDRTFTAEYSFFEGSTKYLAELVELNSFVKSCSFDINAKRTMYRDALDILGIESDKTEEDIEKMIDNILQIDANGDKKLKKNNGLRNFIASNVIDSNRFKYLVRYGNPKKIRETAKCKPAVRFVLNEIPDAQIERYYEACCPKNTALCSANKRREKLADMIAEIKFENFSDAGNYQKANVTSRTSEAEIKRKNQAIIRLYLTVMYIMLKNLVNVNARYVIAFHCVERDTKLYAESGLEVGNIEKNKTNLTMAVMGVKLENGIIKTEFDKSFAENAANRYLRNARWYKLILDNLKKSERAVVNEFANTVCALNAIRNININIKEIKEVENYFALYHYLIQKHLENRFADKKVERDTGDFISKLEEHKTYCKDFVKAYCTPFGYNLVRYKNLTIDGLFDKNYPGKDDSDEQK

SEQ ID NO: 12 - protein, 1437 aa, synthetic construct
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMANDSFAYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELANYIEAFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 13 - protein, 1152 aa, synthetic construct
KVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIAHGIVAFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIANYIAAFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALEG

SEQ ID NO: 14 - protein, 1174 aa, synthetic construct
TEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSAYRHSGSSELPLFDGNMLQRLYNVFDVSVQRVKIDHEHNDEVDPHYHFNHLVRKGKKDRYGHNDNPSFKHHFVDGEGMVTEAGLLFFVSLFLEKRDAIWMQKKIRGFKGGTETYQQMTNEVFCRSRISLPKLKLESLRMDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAERVQGRIKRVIEDVYAVYDAFARDEINTRDELDACLADKGIRRGHLPRQMIAILSQEHKDMEEKIRKKLQEMMADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENRPFLLLKEPKTDRQTLVAGWKGEFHLPRGIFTEAVRDCLIEMGHDEVASYKEVGFMAKAVPLYFERACEDRVQPFYDSPFNVGNSLKPKKGRFLSKEERAEEWERGKERFRDLEAWSYSAARRIEDAFAGIEYASPGNKKKIEQLLRDLSLWEAFESKLKVRADRINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFREMLESWSDPLLAKWPELHGKVRLLIAVRNAFSANQYPMYDEAVFSSIRKYDPSSPDAIEERMGLNIAHRLSEEVKQAKETVERIIQA

SEQ ID NO: 15 - protein, 1089 aa, synthetic construct
NIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMK

SEQ ID NO: 16 - protein, 1094 aa, synthetic construct
EKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLRNYYSAYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCANQYPFYKEALHAPIPLFTVAQPTTEEKDGLGIAEALLKVLREYCEIVKSQI

SEQ ID NO: 17 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 18 - protein, 1490 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 19 - protein, 1495 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSGGGGSQLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKSTEQDQFSLT

SEQ ID NO: 20 - protein, 1495 aa, synthetic construct
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLANYYSAYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVANAFCANQYPFYKEALHAPIPLFTVAQPTTEEKDGLGIAEALLKVLREYCEIVKSQIGSSLQKKLEELELGSQLHLPQVLADAVSRLVIGKFGDLTDNFSSPHARRIGLAGVVMTTGTDVKDAKVICVSTGSKCINGEYLSDRGLALNDCHAEIVSRRSLLRFLYTQLELYLNNEDDQKRSIFQKSERGGFRLKENIQFHLYISTSPCGDARIFSPHEAILEEPADRHPNRKARGQLRTKIEAGQGTIPVRNNASIQTWDGVLQGERLLTMSCSDKIARWNVVGIQGSLLSIFVEPIYFSSIILGSLYHGDHLSRAMYQRISNIEDLPPLYTLNKPLLTGISNAEARQPGKAPIFSVNWTVGDSAIEVINATTGKGELGRASRLCKHALYCRWMRVHGKVPSHLLRSKITKPNVYHETKLAAKEYQAAKARLFTAFIKAGLGAWVEKPTEQDQFSLT

SEQ ID NO: 21 - protein, 1495 aa, synthetic construct
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLANYYSAYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVANAFCANQYPFYKEALHAPIPLFTVAQPTTEEKDGLGIAEALLKVLREYCEIVKSQIGSSLQKKLEELELGSQLHLPQVLADAVSRLVIGKFGDLTDNFSSPHARRIGLAGVVMTTGTDVKDAKVICVSTGAKCINGEYLSDRGLALNDCHAEIVSRRSLLRFLYTQLELYLNNEDDQKRSIFQKSERGGFRLKENIQFHLYISTSPCGDARIFSPHEAILEEPADRHPNRKARGQLRTKIEAGQGTIPVRNNASIQTWDGVLQGERLLTMSCSDKIARWNVVGIQGSLLSIFVEPIYFSSIILGSLYHGDHLSRAMYQRISNIEDLPPLYTLNKPLLTGISNAEARQPGKAPIFSVNWTVGDSAIEVINATTGKGELGRASRLCKHALYCRWMRVHGKVPSHLLRSKITKPNVYHETKLAAKEYQAAKARLFTAFIKAGLGAWVEKPTEQDQFSLT

SEQ ID NO: 22 - protein, 1218 aa, synthetic construct
MKRTADGSEFESPKKKRKVNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSLQLPPLERLTLGSGSSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDDDPLYKEALQMLRDAGAQVSIMTYDEFKHCWDTFVDHQGCPFQPWDGLDEHSQALSGRLRAILQNQGN

SEQ ID NO: 23 - protein, 803 aa, synthetic construct
MKRTADGSEFESPKKKRKVGSIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNGTSKRTADGSEFESPKKKRKVGPGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDDDPLYKEALQMLRDAGAQVSIMTYDEFKHCWDTFVDHQGCPFQPWDGLDEHSQALSGRLRAILQNQGN

SEQ ID NO: 24 - protein, 1218 aa, synthetic construct
MKRTADGSEFESPKAAAKVNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSLQLPPLERLTLGSGSSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDDDPLYKEALQMLRDAGAQVSIMTYDEFKHCWDTFVDHQGCPFQPWDGLDEHSQALSGRLRAILQNQGN

SEQ ID NO: 25 - DNA, 8076 bp, synthetic construct
  source 1..8076: mol_type = other DNA
cgtggagtccaacccagggcccgctagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacccacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacgggcccgcggaattccgtttaagaccaatgacttacaaggcagctgtagatcttagccactttttaaaagaaaaggggggactggaagggctaattcactcccaacgaagacaagatatgatcaaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagtaggacttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgtgccggactggaagggctaattcactcccaacgaagacaagatatgatcaaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccgcccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgtgaaacagactttgaattttgaccttctcaagttggcgggcga